SEQUENCE LISTING (38 sequences)

SEQ ID NO: 1 - DNA, 2637 bp, synthetic construct
  source 1..2637: mol_type = other DNA
atgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagcggtggtggtagcggtggtggtagcggtggtggtagcatgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataa

SEQ ID NO: 2 - DNA, 1896 bp, Escherichia phage N15
  source 1..1896: mol_type = other DNA
atgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaa

SEQ ID NO: 3 - DNA, 56 bp, synthetic construct
  source 1..56: mol_type = other DNA
tatcagcacacaattgcccattatacgcgcgtataatggactattgtgtgctgata

SEQ ID NO: 4 - DNA, 708 bp, Saccharomyces cerevisiae
  source 1..708: mol_type = other DNA
atgaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataa

SEQ ID NO: 5-7 - skipped (3 entries)

SEQ ID NO: 8 - DNA, 80 bp, synthetic construct
  source 1..80: mol_type = other DNA
tagggataacagggtaattttttttatcagcacacaattgcccattatacgcgcgtataatggactattgtgtgctgata

SEQ ID NO: 9-10 - skipped (2 entries)

SEQ ID NO: 11 - DNA, 1143 bp, synthetic construct
  source 1..1143: mol_type = other DNA
gctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 12 - DNA, 55 bp, synthetic construct
  source 1..55: mol_type = other DNA
tacctgacgctttttatcgcaactctctactgtttctccatacccgtttttttgg

SEQ ID NO: 13 - DNA, 299 bp, synthetic construct
  source 1..299: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgg

SEQ ID NO: 14 - DNA, 24 bp, synthetic construct
  source 1..24: mol_type = other DNA
atgagcaaggtaaaaatcggtgag

SEQ ID NO: 15 - DNA, 23 bp, synthetic construct
  source 1..23: mol_type = other DNA
ttagctgtagtacgtttcccatg

SEQ ID NO: 16 - DNA, 30 bp, synthetic construct
  source 1..30: mol_type = other DNA
atgaaaaacatcaaaaaaaaccaggtaatg

SEQ ID NO: 17 - DNA, 23 bp, synthetic construct
  source 1..23: mol_type = other DNA
ttatttcaggaaagtttcggagg

SEQ ID NO: 18 - DNA, 27 bp, synthetic construct
  source 1..27: mol_type = other DNA
gctaatcttatggataaaaatgctatg

SEQ ID NO: 19 - DNA, 24 bp, synthetic construct
  source 1..24: mol_type = other DNA
ttatgacaacttgacggctacatc

SEQ ID NO: 20 - DNA, 26 bp, synthetic construct
  source 1..26: mol_type = other DNA
aagaaaccaattgtccatattgcatc

SEQ ID NO: 21 - DNA, 25 bp, synthetic construct
  source 1..25: mol_type = other DNA
ccaaaaaaacgggtatggagaaaca

SEQ ID NO: 22-23 - skipped (2 entries)

SEQ ID NO: 24 - protein, 631 aa, Escherichia phage N15
MSKVKIGELINTLVNEVEAIDASDRPQGDKTKRIKAAAARYKNALFNDKRKFRGKGLQKRITANTFNAYMSRARKRFDDKLHHSFDKNINKLSEKYPLYSEELSSWLSMPTANIRQHMSSLQSKLKEIMPLAEELSNVRIGSKGSDAKIARLIKKYPDWSFALSDLNSDDWKERRDYLYKLFQQGSALLEELHQLKVNHEVLYHLQLSPAERTSIQQRWADVLREKKRNVVVIDYPTYMQSIYDILNNPATLFSLNTRSGMAPLAFALAAVSGRRMIEIMFQGEFAVSGKYTVNFSGQAKKRSEDKSVTRTIYTLCEAKLFVELLTELRSCSAASDFDEVVKGYGKDDTRSENGRINAILAKAFNPWVKSFFGDDRRVYKDSRAIYARIAYEMFFRVDPRWKNVDEDVFFMEILGHDDENTQLHYKQFKLANFSRTWRPEVGDENTRLVALQKLDDEMPGFARGDAGVRLHETVKQLVEQDPSAKITNSTLRAFKFSPTMISRYLEFAADALGQFVGENGQWQLKIETPAIVLPDEESVETIDEPDDESQDDELDEDEIELDEGGGDEPTEEEGPEEHQPTALKPVFKPAKNNGDGTYKIEFEYDGKHYAWSGPADSPMAAMRSAWETYYS

SEQ ID NO: 25 - protein, 235 aa, Saccharomyces cerevisiae
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 26 - DNA, 32 bp, synthetic construct
  source 1..32: mol_type = other DNA
cataataacaatatcatgatattgttattatg

SEQ ID NO: 27 - DNA, 90 bp, synthetic construct
  source 1..90: mol_type = other DNA
cctatattgggccacctatgtatgcacagttcgcccatactatacgtatagtatgggcgaactgtgcatacataggtggcccaatatagg

SEQ ID NO: 28 - DNA, 90 bp, synthetic construct
  source 1..90: mol_type = other DNA
gggatcccgttccatacatacatgtatccatgtggcatactatacgtatagtatgccgatgttacatatggtatcattcgggatcccgtt

SEQ ID NO: 29 - DNA, 44 bp, synthetic construct
  source 1..44: mol_type = other DNA
cacacaattgcccattatacgcgcgtataatgggcaattgtgtg

SEQ ID NO: 30 - DNA, 42 bp, synthetic construct
  source 1..42: mol_type = other DNA
acctatttcagcatactacgcgcgtagtatgctgaaataggt

SEQ ID NO: 31 - DNA, 18 bp, synthetic construct
  source 1..18: mol_type = other DNA
attaccctgttatcccta

SEQ ID NO: 32 - DNA, 27 bp, synthetic construct
  source 1..27: mol_type = other DNA
taactataacggtcctaaggtagcgaa

SEQ ID NO: 33 - DNA, 22 bp, synthetic construct
  source 1..22: mol_type = other DNA
caaaacgtcgtgagacagtttc

SEQ ID NO: 34 - DNA, 36 bp, synthetic construct
  source 1..36: mol_type = other DNA
ggtggtggtagcggtggtggtagcggtggtggtagc

SEQ ID NO: 35 - DNA, 816 bp, unidentified
  source 1..816: mol_type = unassigned DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 36 - DNA, 20 bp, synthetic construct
  source 1..20: mol_type = other DNA
taacccgggcgtttaaacag

SEQ ID NO: 37 - DNA, 35 bp, synthetic construct
  source 1..35: mol_type = other DNA
gtttaaacgcccgggttagctgtagtacgtttccc

SEQ ID NO: 38 - DNA, 35 bp, synthetic construct
  source 1..35: mol_type = other DNA
gacaattggtttcttattaccctgttatccctaat

(19) United States
(12) Patent Application Publication
(10) Pub. No.: US 2026/0317694 A1
(43) Pub. Date: Oct. 15, 2026

(54) TRANSFORMED CELL AND METHOD FOR PRODUCING LINEAR COVALENTLY CLOSED DNA

(71) Applicant: KANEKA CORPORATION, Osaka (JP)

(72) Inventor: Teruyuki Nishi, Hyogo (JP)

(73) Assignee: KANEKA CORPORATION, Osaka (JP)

(21) Appl. No.: 19/564,868

(22) Filed: Mar. 12, 2026

Related U.S. Application Data
(63) Continuation of application No. PCT/JP2024/029525, filed on Aug. 20, 2024.

(30) Foreign Application Priority Data
Sep. 12, 2023 (JP) 2023-147265

(51) Int. Cl. C12N 1/20; C12N 9/22; C12N 15/70; C12P 19/34; C12R 1/185

(52) U.S. Cl. CPC C12N 1/20; C12N 9/22; C12N 15/70; C12P 19/34; C12R 2001/185

(57) ABSTRACT

Provided is a method for simply and efficiently producing a linear covalently closed DNA. A transformed cell includes two double-stranded circular DNA vectors that encode a protelomerase or an active protelomerase fragment and an endonuclease or an active endonuclease fragment, which are each placed under the control of promoters capable of controlling their expression. The first double-stranded circular DNA vector includes a pair of protelomerase recognition sequences which are recognized and cleaved by protelomerase activity, a nucleic acid sequence of interest between one pair of protelomerase recognition sequences, and at least one endonuclease recognition sequence between the other pair of protelomerase recognition sequences and which is recognized and cleaved by endonuclease activity. The second double-stranded circular DNA vector includes the protelomerase gene sequence and/or the endonuclease gene sequence and at least one endonuclease recognition sequence, which is recognized and cleaved by endonuclease activity.

### SEQUENCE LISTING

This application contains a sequence listing in computer readable form (File name: B230614.xml; date of creation: Feb. 9, 2026; File size: 38,134 bytes) which is incorporated herein by reference in its entirety and forms part of the disclosure.

### TECHNICAL FIELD

One or more embodiments of the present invention relate to a transformed cell and a method for producing a linear covalently closed DNA.

### BACKGROUND ART

Double-stranded circular DNA vectors are widely used as DNA vectors that can be conveniently handled. A double-stranded circular DNA vector comprises not only a gene sequence of interest, but also a replication initiation region, which is a sequence necessary for maintenance and replication of a plasmid, and an antibiotic-resistance gene and the like. When a double-stranded circular DNA vector is introduced into an animal tissue or cell, these sequences are delivered together with the gene sequence of interest. However, this is undesirable from the viewpoint of avoiding immunogenicity.

As a means for obtaining only a gene sequence of interest by removing unnecessary sequences from a double-stranded circular DNA vector, a linear covalently closed DNA (LCC DNA) is used (Patent Documents 1 and 2).

A linear covalently closed DNA is a linear double-stranded DNA whose terminal portion is closed with a hairpin structure, and can be produced by utilizing the activity of a protelomerase that cleaves a double-stranded DNA such that a hairpin structure is formed at the cleaved end. For example, a double-stranded circular DNA vector in which a gene sequence of interest is placed between a pair of protelomerase recognition sequences is provided. Subsequently, by applying the protelomerase activity to this double-stranded circular DNA vector, a linear covalently closed DNA that comprises only the gene sequence of interest and whose end is closed with a hairpin structure can be produced. The thus produced linear covalently closed DNA is stable because of its resistance to an exonuclease, and in addition, has an advantage that it can be efficiently introduced into a cell because of its small size. Further, since the placement of the protelomerase recognition sequences enables removal of the replication initiation region, the antibiotic-resistance gene, and the like from the linear covalently closed DNA comprising the gene of interest, the risks of immunogenicity and the like can also be reduced.

Patent Document 1 discloses a method for producing a linear covalently closed DNA in vitro. The method described in Patent Document 1 is a method in which a double-stranded DNA molecule is synthesized in vitro using a DNA polymerase, and then the double-stranded DNA molecule is cleaved by treatment with a protelomerase to produce the linear covalently closed DNA. However, this method involves a laborious procedure in which various enzymes are reacted in vitro. Furthermore, an unnecessary second linear covalently closed DNA derived from the vector sequence other than the gene sequence of interest is generated simultaneously with a first linear covalently closed DNA comprising the gene sequence of interest, and various isolated enzymes are required in order to degrade and remove the unnecessary second linear covalently closed DNA, which makes the method costly.

Patent Document 2 discloses a method for producing a linear covalently closed DNA in a cell. The method described in Patent Document 2 is a method in which a double-stranded DNA molecule is amplified in a cell, and then the double-stranded DNA molecule is cleaved by inducing expression of a protelomerase in the cell to produce the linear covalently closed DNA. However, also in this method, an unnecessary second linear covalently closed DNA derived from the vector sequence other than the gene sequence of interest is generated simultaneously with a first linear covalently closed DNA comprising the gene sequence of interest. Therefore, after the first and second linear covalently closed DNAs are isolated from Escherichia coli, a procedure for selectively degrading and removing the second linear covalently closed DNA in vitro is still necessary.

Accordingly, a novel method for producing a linear covalently closed DNA more conveniently and efficiently is required.

### CITATION LIST

### Patent Literature

Patent Literature 1: U.S. Pat. No. 9,109,250 B
Patent Literature 2: WO 2022/209987.

### SUMMARY

One or more embodiments of the present invention aim to provide a novel method for simply and efficiently producing a linear covalently closed DNA.

The reaction of cleaving a double-stranded circular DNA vector by the protelomerase activity to generate a linear covalently closed DNA results in generation of not only a first linear covalently closed DNA comprising a gene of interest, but also an unnecessary second linear covalently closed DNA that is a linear covalently closed DNA derived from the vector sequence other than the gene of interest.

In order to solve the above-described problem, the present inventors conceived a method in which elements required for generating a linear covalently closed DNA in a cell are introduced into two or more double-stranded circular DNA vectors to achieve convenient production of a linear covalently closed DNA. Specifically, in order to generate a linear covalently closed DNA of interest from a first double-stranded circular DNA vector in a cell and, at the same time, induce degradation of an unnecessary linear covalently closed DNA produced as a by-product, a gene of interest, which is positioned between protelomerase recognition sequences, and an endonuclease recognition sequence were placed in a first double-stranded circular DNA vector, and a protelomerase gene sequence and/or an endonuclease gene sequence, and an endonuclease recognition sequence were placed in a second double-stranded circular DNA vector. The endonuclease and the endonuclease recognition sequences were introduced in order to induce degradation of the unnecessary linear covalently closed DNA produced as the above-described by-product and the second double-stranded circular DNA vector.

The present inventors introduced the above-described first and second double-stranded circular DNA vectors into Escherichia coli as a host cell and induced expression of the protelomerase and the endonuclease, and found that, by inducing degradation of the unnecessary linear covalently closed DNA produced as a by-product and the second double-stranded circular DNA vector, a linear covalently closed DNA comprising the gene sequence of interest can be conveniently and efficiently produced. One or more embodiments of the present invention are based on the above-described findings and provides the following.

(1) A transformed cell comprising a first double-stranded circular DNA vector and a second double-stranded circular DNA vector, comprising:
a protelomerase gene sequence encoding a protelomerase or an active fragment thereof which is placed under the control of a promoter capable of controlling expression; and
an endonuclease gene sequence encoding an endonuclease or an active fragment thereof which is placed under the control of a promoter capable of controlling expression,
wherein the first double-stranded circular DNA vector comprises
a pair of protelomerase recognition sequences which are recognized and cleaved by the protelomerase or an active fragment thereof,
a nucleic acid sequence of interest which is placed in a region between the pair of protelomerase recognition sequences, and
at least one endonuclease recognition sequence which is placed in the other region between the pair of protelomerase recognition sequences and which is recognized and cleaved by the endonuclease or an active fragment thereof, and
wherein the second double-stranded circular DNA vector comprises the protelomerase gene sequence and/or the endonuclease gene sequence, and comprises at least one endonuclease recognition sequence which is recognized and cleaved by the endonuclease or an active fragment thereof.

(2) The transformed cell of (1), wherein two or more double-stranded circular DNA vectors comprising the first double-stranded circular DNA vector and the second double-stranded circular DNA vector comprise the protelomerase gene sequence and the endonuclease gene sequence.

(3) The transformed cell of (2), wherein the first double-stranded circular DNA vector comprises the protelomerase gene sequence in the other region between the pair of protelomerase recognition sequences, and the second double-stranded circular DNA vector comprises the endonuclease gene sequence.

(4) The transformed cell of (2), wherein the first double-stranded circular DNA vector comprises the endonuclease gene sequence in the other region between the pair of protelomerase recognition sequences, and the second double-stranded circular DNA vector comprises the protelomerase gene sequence.

(5) The transformed cell of (2), wherein the second double-stranded circular DNA vector comprises the protelomerase gene sequence and the endonuclease gene sequence.

(6) The transformed cell of (2), wherein the transformed cell further comprises a third double-stranded circular DNA vector,
wherein the second double-stranded circular DNA vector comprises the protelomerase gene sequence, and
wherein the third double-stranded circular DNA vector comprises
the endonuclease gene sequence, and
at least one endonuclease recognition sequence which is recognized and cleaved by the endonuclease or an active fragment thereof.

(7) The transformed cell of (2), wherein
the second double-stranded circular DNA vector comprises the protelomerase gene sequence and the endonuclease gene sequence, and
the protelomerase gene sequence and the endonuclease gene sequence encode a fusion polypeptide comprising the protelomerase or an active fragment thereof and the endonuclease or an active fragment thereof.

(8) The transformed cell of any one of (1) to (7), wherein the first double-stranded circular DNA vector further comprises a spacer sequence placed between the endonuclease recognition sequence and the protelomerase recognition sequence.

(9) The transformed cell of any one of (1) to (7), wherein the protelomerase is TelN protelomerase, TelA protelomerase, or TelK protelomerase.

(10) The transformed cell of any one of (1) to (7), wherein the endonuclease is a homing endonuclease.

(11) The transformed cell of (10), wherein the homing endonuclease is I-SceI homing endonuclease, I-CeuI homing endonuclease, or I-CreI homing endonuclease.

(12) The transformed cell of any one of (1) to (7), wherein the first double-stranded circular DNA vector and the second double-stranded circular DNA vector comprise antibiotic-resistance genes which provide resistance to different antibiotics.

(13) The transformed cell of any one of (1) to (7), wherein the first double-stranded circular DNA vector and the second double-stranded circular DNA vector are replicated by an identical replication mechanism.

(14) The transformed cell of (13), wherein the first double-stranded circular DNA vector and the second double-stranded circular DNA vector comprise an identical replication initiation region.

(15) The transformed cell of any one of (1) to (7), wherein the cell is a bacterial cell or a eukaryotic cell.

(16) The transformed cell of (15), wherein the bacterium is Escherichia or Bacillus.

(17) The transformed cell of any one of (1) to (7), wherein the transformed cell expresses an exonuclease.

(18) The transformed cell of any one of (1) to (7), wherein the nucleic acid sequence of interest comprises a gene sequence of interest encoding a protein of interest or a fragment thereof.

(19) A composition for transformation, comprising at least a first double-stranded circular DNA vector and a second double-stranded circular DNA vector,
wherein the first double-stranded circular DNA vector comprises
a pair of protelomerase recognition sequences,
a nucleic acid sequence of interest which is placed in a region between the pair of protelomerase recognition sequences, and
at least one endonuclease recognition sequence which is placed in the other region between the pair of protelomerase recognition sequences,
wherein the second double-stranded circular DNA vector comprises
a protelomerase gene sequence which encodes a protelomerase or an active fragment thereof that recognizes and cleaves the pair of protelomerase recognition sequences and which is placed under the control of a promoter capable of controlling expression, and/or
an endonuclease gene sequence which encodes an endonuclease or an active fragment thereof that recognizes and cleaves the endonuclease recognition sequence and which is placed under the control of a promoter capable of controlling expression, and
wherein the second double-stranded circular DNA vector comprises at least one endonuclease recognition sequence which is recognized and cleaved by the endonuclease or an active fragment thereof.

(20) A kit for transformation, comprising at least a first double-stranded circular DNA vector and a second double-stranded circular DNA vector,
wherein the first double-stranded circular DNA vector comprises
a pair of protelomerase recognition sequences,
a nucleic acid sequence of interest or a multi-cloning site for introducing the same which is placed in a region between the pair of protelomerase recognition sequences, and
at least one endonuclease recognition sequence which is placed in the other region between the pair of protelomerase recognition sequences,
wherein the second double-stranded circular DNA vector comprises
a protelomerase gene sequence which encodes a protelomerase or an active fragment thereof that recognizes and cleaves the pair of protelomerase recognition sequences and which is placed under the control of a promoter capable of controlling expression, and/or
an endonuclease gene sequence which encodes an endonuclease or an active fragment thereof that recognizes and cleaves the endonuclease recognition sequence and which is placed under the control of a promoter capable of controlling expression, and
wherein the second double-stranded circular DNA vector comprises at least one endonuclease recognition sequence which is recognized and cleaved by the endonuclease or an active fragment thereof.

(21) A method for producing a linear covalently closed DNA, comprising:
culturing the transformed cell of any one of (1) to (7);
inducing the expression of the protelomerase or an active fragment thereof and the endonuclease or an active fragment thereof in the transformed cell after the culturing step; and
extracting DNA from the transformed cell after the inducing step.

(22) The method of (21), further comprising isolating the linear covalently closed DNA after the extracting step.

(23) The method of (21), further comprising
introducing the composition for transformation of (19) to a host cell so as to produce the transformed cell comprising the first double-stranded circular DNA vector and the second double-stranded circular DNA vector, before the culturing step.

(24) The method of (21), further comprising
introducing the first double-stranded circular DNA vector to the host cell transformed with the second double-stranded circular DNA vector so as to produce the transformed cell comprising the first double-stranded circular DNA vector and the second double-stranded circular DNA vector, before the culturing step.

The present description encompasses the content of disclosure of Japanese Patent Application No. 2023-147265, which forms the basis of the priority of the present application.

### Advantageous Effects

According to one or more embodiments of the present invention, a method for conveniently and efficiently producing a linear covalently closed DNA is provided.

### BRIEF DESCRIPTION OF DRAWINGS

FIG. 1A schematically shows the first and second double-stranded circular DNA vectors in nonlimiting Embodiment (i) in the present description.

FIG. 1B schematically shows the first and second double-stranded circular DNA vectors in nonlimiting Embodiment (ii) in the present description.

FIG. 2A schematically shows the first and second double-stranded circular DNA vectors in nonlimiting Embodiment (iii) in the present description.

FIG. 2B schematically shows the first to third double-stranded circular DNA vectors in nonlimiting Embodiment (iv) in the present description.

FIG. 3 shows a Comparative Example vector for producing a linear covalently closed DNA.

FIG. 4 shows a control vector (T6), which is one of the two vectors produced in Example 1 for producing a linear covalently closed DNA.

FIG. 5 shows an I-SceI vector, which is one of the two vectors produced in Example 1 for producing a linear covalently closed DNA.

FIG. 6 shows the results of electrophoresis of linear covalently closed DNAs in Example 2. “Linear covalently closed DNA of interest” represents a linear covalently closed DNA comprising a nucleic acid encoding a packaging protein, and “unnecessary linear covalently closed DNA” represents other linear covalently closed DNAs. In the figure, the bands with higher mobility correspond to the remaining I-SceI vector. “M” represents a DNA marker (N3200, manufactured by New England Biolabs).

### DETAILED DESCRIPTION

### 1. Transformed Cell

### 1-1. Overview

A first aspect of one or more embodiments of the present invention is a transformed cell. The transformed cell of the present aspect comprises two or more double-stranded circular DNA vectors. Specifically, the transformed cell of the present aspect comprises a first double-stranded circular DNA vector comprising a nucleic acid sequence of interest placed in a region between a pair of protelomerase recognition sequences, and a second double-stranded circular DNA vector comprising a protelomerase gene sequence and/or an endonuclease gene sequence. The transformed cell of the present aspect enables convenient and efficient production of a linear covalently closed DNA comprising a nucleic acid sequence of interest.

### 1-2. Definition of Terms

Definitions are provided for the following terms, which are frequently used in the present description.

In the present description, “linear covalently closed DNA (LCC DNA)” refers to a linear double-stranded DNA whose ends are closed by hairpin structures. At each end of the linear covalently closed DNA, a 5′-end and a 3′-end of the two DNA strands are linked to form a hairpin structure. In the linear covalently closed DNA, the end portion protected by the hairpin structure has resistance to degradation by an exonuclease.

In the present description, “protelomerase” means a polypeptide having the activity of recognizing a specific sequence in a double-stranded DNA molecule, cleaving the double-stranded DNA molecule at a position in or near the recognition sequence, and linking the 5′ end and the 3′ end at each cleaved end to form a covalently closed end (hereinafter referred to as “protelomerase activity”). In the present description, the type of the protelomerase is not limited as long as the protelomerase has the above-described activity. Examples of the protelomerase include a protelomerase derived from Agrobacterium fabrum (TelA protelomerase, ACCESSION No. AAK88254), a protelomerase derived from the Halomonas virus HAP1 (ACCESSION No. ABY90402), a protelomerase derived from the Vibrio virus VP882 (ACCESSION No. ABM73418), a protelomerase derived from the Klebsiella phage phiKO2 (TelK protelomerase, ACCESSION No. AAR83042), a protelomerase derived from Rhizobium pusense (ACCESSION No. QKJ91773), a protelomerase derived from a Feldmannia species virus (ACCESSION No. ACH46812), a protelomerase derived from the Vibrio phage vB_VpaM_MAR (ACCESSION No. AFV81380), a protelomerase derived from the Yersinia phage PY54 (Tel protelomerase, ACCESSION No. CAD91792), a protelomerase derived from the Escherichia virus N15 (TelN protelomerase, ACCESSION No. AAB81106), or a mutant of any of the above-described protelomerases. In the present description, the protelomerase is not included in the endonuclease described below.

In the present description, “active fragment” of a protelomerase herein is a polypeptide fragment that comprises a partial region of the protelomerase, wherein the fragment retains the protelomerase activity, for example, at not less than 1%, not less than 10%, not less than 20%, not less than 30%, not less than 40%, not less than 50%, not less than 60%, not less than 70%, not less than 80%, not less than 90%, not less than 95%, not less than 98%, or not less than an equivalent or higher level compared to the activity of the full-length protein. The amino acid length of the polypeptide constituting the active fragment is not limited. For example, the active fragment may be of a region of at least 100, 150, 200, 250, 300, 350, or 400 consecutive amino acids comprising a catalytic domain in the protelomerase.

In the present description, “protelomerase gene” is a gene encoding a protelomerase or an active fragment thereof. Examples of the protelomerase gene include a gene encoding any of the above-described protelomerases or an active fragment thereof. Examples of the protelomerase gene include a protelomerase gene encoding TelN protelomerase consisting of the amino acid sequence of SEQ ID NO:24, such as a TelN protelomerase gene consisting of the base sequence of SEQ ID NO:2.

In the present description, “protelomerase recognition sequence” means a sequence recognized by a protelomerase. A protelomerase recognizes a protelomerase recognition sequence, cleaves a double-stranded DNA molecule at a position in or near the protelomerase recognition sequence, and rejoins the cleaved ends. A protelomerase recognition sequence is known to be a palindrome sequence or a palindrome-like sequence. A pair of sequences constituting a palindrome sequence or a palindrome-like sequence may be referred to, for example, as “telR” and “telL”, and a palindrome sequence or a palindrome-like sequence composed of telR and tell may be referred to as “telRL”. In this case, “protelomerase recognition sequence” refers to a palindrome sequence or a palindrome-like sequence composed of telR and TelL, and “a pair of protelomerase recognition sequences” refers to a pair (that is, two sets) of telRL.

In the present description, “endonuclease” means an enzyme having the activity of cleaving a phosphodiester bond inside a nucleotide chain. Among endonucleases, an endonuclease that recognizes a specific sequence and cleaves a double-stranded DNA molecule in or near the sequence is particularly referred to as a restriction enzyme. Examples of restriction enzymes include not only naturally occurring restriction enzymes, but also artificial restriction enzymes such as TALEN and zinc finger nuclease (ZFN), genome editing enzymes such as Cas9 protein, or a mutant of any of the above-described endonucleases. The endonuclease may be either an enzyme having the activity of cleaving the two DNA strands or an enzyme that cleaves only one DNA strand (nickase).

Among restriction enzymes, those found in inteins, introns, and the like are particularly referred to as “homing endonucleases”. A homing endonuclease is generally known to recognize a recognition sequence of as long as 12 to 40 bp, and the recognition sequence is known not to be a palindrome sequence. In the present description, the type of the homing endonuclease is not limited. Examples of the type of the homing endonuclease include I-SceI (ACCESSION No. NP009324), I-CeuI (ACCESSION No. CAA78934), I-CreI (ACCESSION No. CAA26008), I-AniI, I-Chul, I-DmoI, I-CsmI, PI-SceI, PI-TIiI, PI-MtuI, I-SceII, I-SceIII, HO, PI-CivI, PI-CtrI, PI-Aael, PI-Bsul, PI-Dhal, PI-Dral, PI-MavI, PI-MchI, PI-Mful, PI-MflI, PI-MgaI, PI-Mgol, PI-MinI, PI-Mkal, PI-Mmal, PI-MshI, PI-MsmI, I-MsoI, PI-MthI, PI-MtuI, PI-Mxel, PI-NpuI, PI-PfuI, PI-Rmal, PI-SpbI, PI-SspI, PI-FacI, PI-Mjal, PI-Phol, PI-TagI, PI-ThyI, PI-TkoI, PI-TspI, or a mutant of any of the above-described homing endonucleases. Among these, I-SceI, I-Chul, I-DmoI, I-CreI, I-CsmI, PI-PfuI, PI-SceI, PI-TliI, I-MsoI, PI-MtuI, I-CeuI, I-SceII, I-SceIII, HO, a mutant of any of the above-described homing endonucleases, or the like is particularly preferred; I-SceI, I-Chul, I-DmoI, I-CreI, I-CsmI, PI-SceI, PI-PfuI, PI-TliI, I-MsoI, PI-MtuI, I-CeuI, a mutant of any of the above-described homing endonucleases, or the like is more particularly preferred; and I-SceI, I-CeuI, I-CreI, a mutant of any of the above-described homing endonucleases, or the like is still more particularly preferred.

In the present description, “active fragment” of an endonuclease refers to a polypeptide fragment that comprises a partial region of the endonuclease, wherein the fragment retains the endonuclease activity, for example, at not less than 1%, not less than 10%, not less than 20%, not less than 30%, not less than 40%, not less than 50%, not less than 60%, not less than 70%, not less than 80%, not less than 90%, not less than 95%, not less than 98%, or not less than an equivalent or higher level compared to the activity of the full-length protein. The amino acid length of the polypeptide constituting the active fragment is not particularly limited. For example, the active fragment may be of a region of at least 100, 150, 200, 250, 300, 350, or 400 consecutive amino acids comprising a catalytic domain in the endonuclease.

In the present description, “endonuclease gene” refers to a gene encoding an endonuclease or an active fragment thereof. Examples of the endonuclease gene include a gene encoding any of the above-described endonucleases or an active fragment thereof. The endonuclease gene is, for example, a gene encoding an I-SceI homing endonuclease consisting of the amino acid sequence of SEQ ID NO: 25, such as an I-SceI homing endonuclease gene consisting of the base sequence of SEQ ID NO: 4.

In the present description, “endonuclease recognition sequence” means a sequence recognized by an endonuclease. An endonuclease recognizes an endonuclease recognition sequence and cleaves a double-stranded DNA molecule at a position in or near the endonuclease recognition sequence.

In one or more embodiments of the present invention, “double-stranded circular DNA vector” means a circular double-stranded DNA molecule that is capable of being maintained and/or replicated in a cell. The double-stranded circular DNA vector may comprise a sequence necessary for its maintenance or replication in a cell, such as a replication initiation region and/or a gene encoding an antibiotic-resistance protein. The double-stranded circular DNA vector may be, for example, a plasmid vector or a bacmid vector. The vector may also be a shuttle vector capable of being replicated in both a bacterium such as Escherichia coli and a mammalian cell.

### 1-3. Configuration

A transformed cell of the present aspect comprises, as essential components, two or more double-stranded circular DNA vectors, and a protelomerase gene sequence and an endonuclease gene sequence. In the two or more double-stranded circular DNA vectors comprised in the transformed cell of the present aspect, at least one double-stranded circular DNA vector comprises a nucleic acid sequence of interest, and at least one other double-stranded circular DNA vector comprises the above-described protelomerase gene sequence and/or the above-described endonuclease gene sequence. In the present description, a double-stranded circular DNA vector comprising a nucleic acid sequence of interest is referred to as “first double-stranded circular DNA vector,” and a double-stranded circular DNA vector comprising a protelomerase gene sequence and/or an endonuclease gene sequence is referred to as “second double-stranded circular DNA vector” to distinguish them.

In the transformed cell of the present aspect, the protelomerase gene sequence encodes a protelomerase or an active fragment thereof and is placed under the control of a promoter capable of controlling expression. The endonuclease gene sequence encodes an endonuclease or an active fragment thereof and is placed under the control of a promoter capable of controlling expression.

In the present description, “promoter capable of controlling expression” refers to a gene expression regulatory region capable of controlling the expression of a gene or the like placed downstream (in the 3′-end side) thereof in a cell into which a double-stranded circular DNA vector has been introduced, wherein the promoter is capable of inducing the expression of a gene of interest or the like in the cell at an arbitrary time. The promoter capable of controlling expression can be induced, for example, depending on a condition such as the presence or absence of a specific factor, the temperature, or the like. The type of promoter capable of controlling expression is not limited, and may be appropriately selected depending on the purpose. Examples of the promoter capable of controlling expression include a heat-inducible promoter (such as lambda PR and lambda PL), an arabinose-inducible promoter (such as an araBAD promoter), an IPTG-inducible promoter (such as a LAC promoter, a LACUV5 promoter, a TAC promoter, a Trc promoter, and an LPP promoter), a T7 promoter, a low-temperature-inducible promoter (such as a cspA promoter), a Trp promoter, a rhamnose-inducible promoter (such as an rhaT promoter), a proU promoter, a prpB promoter, a phoA promoter, a recA promoter, a tetA promoter, a cadA promoter, or a mutant thereof. An IPTG-inducible promoter and an arabinose-inducible promoter are particularly preferred.

In the present description, “placed under the control of a promoter capable of controlling expression” means that a gene or the like to be expressed is placed in a region that is positioned downstream of, and under the control of, the promoter capable of controlling expression.

In one or more embodiments, in cases where the second double-stranded circular DNA vector comprises both a protelomerase gene sequence and an endonuclease gene sequence, the protelomerase gene sequence and the endonuclease gene sequence may be placed under the control of the same promoter. For example, the protelomerase gene sequence and the endonuclease gene sequence may be a fusion gene sequence encoding a fusion polypeptide comprising a protelomerase and an endonuclease. Further, a gene sequence encoding a self-cleaving peptide such as a 2A peptide may be placed between the protelomerase gene sequence and the endonuclease gene sequence.

In the transformed cell of the present aspect, the type of the protelomerase or an active fragment thereof encoded by the protelomerase gene sequence is not particularly limited as long as the protelomerase or the active fragment thereof has the protelomerase activity, and may be one that does not recognize a sequence other than the pair of protelomerase recognition sequences, particularly does not recognize the nucleic acid sequence of interest, in the first double-stranded circular DNA vector. Further, the same protelomerase recognition sequence may be absent in the genome sequence of the cell into which the first and second double-stranded circular DNA vectors are introduced. The length of the protelomerase recognition sequence recognized by the protelomerase or an active fragment thereof is not limited, and may be not less than 5 bp, not less than 6 bp, not less than 7 bp, not less than 8 bp, not less than 9 bp, not less than 10 bp, not less than 15 bp, not less than 20 bp, not less than 25 bp, not less than 30 bp, not less than 35 bp, not less than 40 bp, not less than 45 bp, not less than 50 bp, not less than 55 bp, not less than 60 bp, not less than 65 bp, not less than 70 bp, not less than 80 bp, or not less than 90 bp. Particularly preferred examples among the above-described protelomerases include TelN protelomerase, TelA protelomerase, or TelK protelomerase, or a mutant thereof. The mutant is not particularly limited, and may be appropriately selected depending on the purpose. The mutant may have a homology of, for example, not less than 80%, not less than 90%, not less than 95%, or not less than 99% to the original amino acid sequence.

In the transformed cell of the present aspect, the endonuclease or an active fragment thereof encoded by the endonuclease gene sequence is not particularly limited as long as the endonuclease or an active fragment thereof has the endonuclease activity, and may be one that does not recognize a sequence other than the endonuclease recognition sequence, particularly does not recognize the nucleic acid sequence of interest, in the double-stranded circular DNA vector. Further, the same endonuclease recognition sequence may be absent in the genome sequence of the cell into which the double-stranded circular DNA vector is introduced. The endonuclease or an active fragment thereof may be a restriction enzyme, or may be a nickase. The length of the endonuclease recognition sequence recognized by the endonuclease or an active fragment thereof is not limited, and may be, for example, not less than 4 bp, not less than 5 bp, not less than 6 bp, not less than 7 bp, not less than 8 bp, not less than 9 bp, not less than 10 bp, not less than 15 bp, not less than 20 bp, not less than 25 bp, not less than 30 bp, not less than 35 bp, not less than 40 bp, not less than 45 bp, not less than 50 bp, not less than 60 bp, not less than 70 bp, not less than 80 bp, not less than 90 bp, or not less than 100 bp.

In one or more embodiments of the present invention, the endonuclease may be a homing endonuclease. Examples of the homing endonuclease include, but are not limited to, I-SceI, I-CeuI, I-CreI, I-AniI, I-Chul, I-DmoI, I-CsmI, PI-SceI, PI-TIiI, PI-MtuI, I-CeuI, I-SceII, I-SceIII, HO, PI-CivI, PI-CtrI, PI-Aael, PI-Bsul, PI-Dhal, PI-Dral, PI-MavI, PI-MchI, PI-Mful, PI-MflI, PI-MgaI, PI-Mgol, PI-MinI, PI-Mkal, PI-Mkal, PI-Mmal, PI-MshI, PI-MsmI, I-MsoI, PI-MthI, PI-MtuI, PI-Mxel, PI-NpuI, PI-PfuI, PI-Rmal, PI-SpbI, PI-SspI, PI-FacI, PI-Mjal, PI-Phol, PI-TagI, PI-ThyI, PI-TkoI, PI-TspI, or a variant thereof. The mutant is not particularly limited, and may be appropriately selected depending on the purpose. The mutant may have a homology of, for example, not less than 80%, not less than 90%, not less than 95%, or not less than 99% to the original amino acid sequence.

In the transformed cell of the present aspect, the first double-stranded circular DNA vector comprises a pair of protelomerase recognition sequences, a nucleic acid sequence of interest which is placed in a region between the pair of protelomerase recognition sequences, and at least one endonuclease recognition sequence which is placed in the other region between the pair of protelomerase recognition sequences. Here, the pair of protelomerase recognition sequences are sequences which are recognized, cleaved, and ligated by the protelomerase or an active fragment thereof encoded by the above-described protelomerase gene sequence. The endonuclease recognition sequence is a sequence which is recognized and cleaved by the endonuclease or an active fragment thereof encoded by the above-described endonuclease gene sequence.

In the transformed cell of the present aspect, the second double-stranded circular DNA vector comprises the above-described protelomerase gene sequence and/or the above-described endonuclease gene sequence, and comprises at least one endonuclease recognition sequence which is recognized and cleaved by the above-described endonuclease or an active fragment thereof. In the transformed cell of the present aspect, at least one of the protelomerase gene sequence or the endonuclease gene sequence may be comprised in the second double-stranded circular DNA vector, or both the protelomerase gene sequence and the endonuclease gene sequence may be comprised in the second double-stranded circular DNA vector. In cases where only one of the protelomerase gene sequence or the endonuclease gene sequence is comprised in the second double-stranded circular DNA vector, the other gene sequence, not comprised in the second double-stranded circular DNA vector, may be comprised in the first double-stranded circular DNA vector, may be comprised in a third double-stranded circular DNA vector, which is a vector other than the first and second double-stranded circular DNA vectors, or may be present in the host genome.

In the transformed cell of the present aspect, the pair of protelomerase recognition sequences in the first double-stranded circular DNA vector are protelomerase recognition sequences recognized by the protelomerase or an active fragment thereof encoded by the protelomerase gene sequence in the transformed cell of the present aspect. Specific examples of the protelomerase recognition sequences include a base sequence recognized by TelA protelomerase (SEQ ID NO: 26), a base sequence recognized by a protelomerase derived from the Halomonas virus HAP1 (SEQ ID NO: 27), a base sequence recognized by a protelomerase derived from the Vibrio virus VP882 (SEQ ID NO: 28), a base sequence recognized by TelK protelomerase (SEQ ID NO: 29), a base sequence recognized by a protelomerase derived from Rhizobium pusense, a base sequence recognized by a protelomerase derived from a Feldmannia species virus, a base sequence recognized by a protelomerase derived from the Vibrio phage vB_VpaM_MAR, a base sequence recognized by Tel protelomerase (SEQ ID NO: 30), and a base sequence recognized by TelN protelomerase (telRL sequence) (SEQ ID NO: 3).

In the transformed cell of the present aspect, the endonuclease recognition sequences in the first double-stranded circular DNA vector and the second double-stranded circular DNA vector are endonuclease recognition sequences recognized by the endonuclease or an active fragment thereof encoded by the endonuclease gene sequence in the transformed cell of the present aspect. Examples of the endonuclease recognition sequences include restriction enzyme recognition sequences, TALEN recognition sequences, ZFN recognition sequences, and CRISPR/Cas9 recognition sequences. In cases where the endonuclease is a homing endonuclease, examples of the homing endonuclease recognition sequence include an I-SceI recognition sequence (SEQ ID NO: 31), an I-CeuI recognition sequence (SEQ ID NO: 32), and an I-CreI recognition sequence (SEQ ID NO: 33). The endonuclease recognition sequences in the first double-stranded circular DNA vector and the second double-stranded circular DNA vector may be sequences recognized by the same endonuclease (for example, endonuclease recognition sequences consisting of the same base sequence), or may be sequences recognized by different endonucleases (for example, endonuclease recognition sequences consisting of different base sequences).

In the transformed cell of the present aspect, the number of endonuclease recognition sequences in the first double-stranded circular DNA vector and the second double-stranded circular DNA vector may be one or more. For example, the first double-stranded circular DNA vector and/or the second double-stranded circular DNA vector may comprise 1, 2, 3, 4, 5, 6, 7, 8, 9, or not less than 10 endonuclease recognition sequences. However, in the first double-stranded circular DNA vector, the region comprising the nucleic acid sequence of interest between the pair of protelomerase recognition sequences does not comprise an endonuclease recognition sequence.

In the present description, “nucleic acid sequence of interest” is not particularly limited as long as it is a sequence comprising not less than two nucleotides. More specifically, the nucleic acid sequence of interest is not limited to a sequence encoding a protein or a fragment thereof, or encoding an RNA. For example, the nucleic acid sequence of interest may be a sequence capable of functioning as a nucleic acid pharmaceutical, or may be a sequence that can be used for introducing a nucleic acid sequence of interest. Specific examples of the nucleic acid sequence include, but are not limited to, a gene sequence encoding a protein or a fragment thereof; a nucleic acid sequence encoding a non-coding RNA such as an siRNA, an shRNA, an miRNA, an lncRNA, or a ribozyme; or a nucleic acid pharmaceutical such as an antisense nucleic acid, an aptamer, or a bait nucleic acid. Examples of the sequence that can be used for introducing a nucleic acid sequence of interest include a sequence capable of being recognized/cleaved by a nuclease such as a restriction enzyme or a genome editing enzyme, and a multi-cloning site comprising a sequence capable of being recognized/cleaved by not less than two nucleases.

In the present description, “gene sequence of interest” means a gene sequence encoding a protein of interest or a fragment thereof, or encoding an RNA molecule of interest. The protein of interest or a fragment thereof is not particularly limited, and may be appropriately selected depending on the purpose. Examples of the protein of interest or a fragment thereof include a polypeptide constituting a virus; a polypeptide produced by an animal, a plant, a fungus, an alga, a bacterium, a virus, or the like; and fragments thereof. These can be used as cell/gene therapeutic agents, vaccines, therapeutic agents for diseases, and the like. The polypeptide produced by an animal, a plant, a fungus, an alga, a bacterium, a virus, or the like is not particularly limited, and may be appropriately selected depending on the purpose. Examples of such a polypeptide include enzymes such as phytase, amylase, glucosidase, cellulase, lipase, protease, glutaminase, peptidase, oxidase, lactase, xylanase, trypsin, pectinase, and isomerase; antibody-binding proteins such as protein A, protein G, and protein L; antibodies or antibody fragments such as a human antibody, a humanized antibody, a chimeric antibody, a llama antibody, an alpaca antibody, a single-chain antibody, a heavy-chain antibody, a multivalent antibody, Fab, F(ab′), F(ab′)₂, Fc, an Fc-fusion protein, a bispecific antibody, a heavy chain (H chain), a light chain (L chain), single-chain Fv (scFv), sc(Fv)₂, disulfide-bonded Fv (sdFv), a diabody, and an antibody-like molecule-targeting peptide (microantibody), and other functional proteins and the like; conjugates (antibody-like molecules) of an antibody fragment and a compound other than an antibody or an antibody fragment; serum albumin such as human serum albumin; an epithelial growth factor such as human epithelial growth factor; insulin; growth hormone; erythropoietin; interferon; blood coagulation factor VIII; granulocyte colony-stimulating factor (G-CSF); granulocyte-macrophage colony-stimulating factor (GM-CSF); thrombopoietin; IL-1; IL-6; tissue plasminogen activator (TPA); urokinase; leptin; stem cell factor (SCF); fibroin; a fluorescent protein; hepatitis B virus surface antigen; and hirudin.

The nucleic acid sequence of interest such as the gene sequence of interest may comprise a promoter in addition to the nucleic acid sequence of interest (for example, a sequence encoding a protein of interest or a fragment thereof). The nucleic acid sequence of interest may further comprise components such as an intron, an enhancer, a terminator, and/or a poly(A) signal, when necessary.

The base length of the nucleic acid sequence of interest is not particularly limited as long as the double-stranded circular DNA vector can accommodate the nucleic acid sequence of interest. For example, the base length may be not less than 6 bases, not less than 10 bases, not less than 20 bases, not less than 50 bases, not less than 100 bases, not less than 200 bases, not less than 500 bases, not less than 1000 bases, not less than 2000 bases, not less than 5000 bases, not less than 10,000 bases, not less than 12,000 bases, not less than 15,000 bases, or not less than 20,000 bases, and/or may be not more than 30,000 bases, not more than 25,000 bases, not more than 20,000 bases, not more than 15,000 bases, not more than 12,000 bases, not more than 10,000 bases, or not more than 5000 bases. Examples of ranges of the base length include 5000 bases to 30,000 bases, 10,000 bases to 25,000 bases, 12,000 bases to 20,000 bases, or 15,000 bases to 18,000 bases. In the transformed cell of the present aspect, the first double-stranded circular DNA vector is advantageous because the vector can accommodate a sequence ranging from an extremely short sequence such as a restriction enzyme recognition sequence to a nucleic acid sequence of interest having a large size.

In some embodiments, in the transformed cell of the present aspect, two or more double-stranded circular DNA vectors comprising the first double-stranded circular DNA vector and the second double-stranded circular DNA vector comprise a protelomerase gene sequence and an endonuclease gene sequence. In the present description, “two or more double-stranded circular DNA vectors” may be, for example, two to five, two to four, or two to three double-stranded circular DNA vectors, or may be two double-stranded circular DNA vectors or three double-stranded circular DNA vectors.

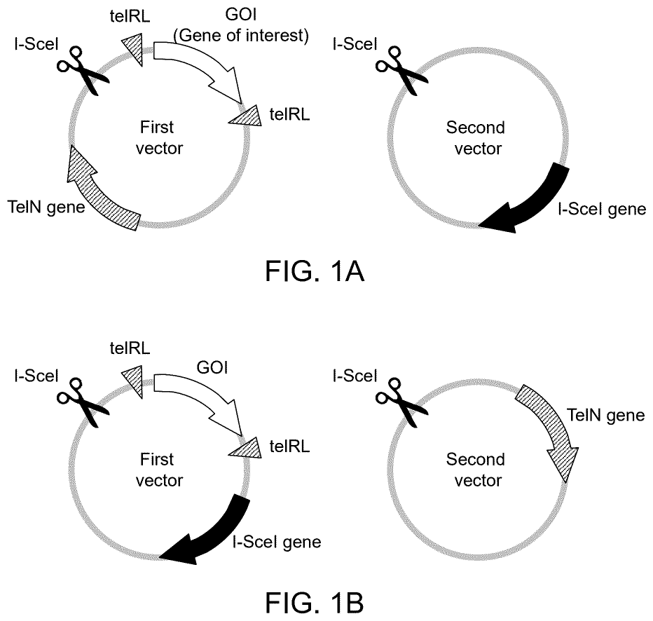

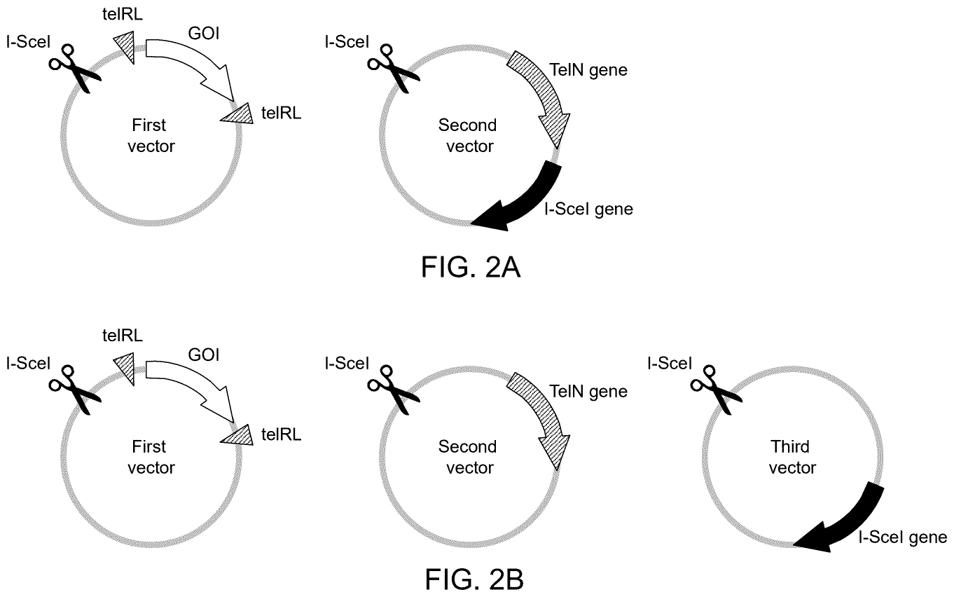

Further embodiments of the transformed cell of the present aspect include the following embodiments (i) to (iv). The following embodiments are schematically illustrated in FIGS. 1A, 1B, 2A, and 2B.

Nonlimiting Embodiment (i): The first double-stranded circular DNA vector comprises the protelomerase gene sequence, and the second double-stranded circular DNA vector comprises the endonuclease gene sequence. In the present nonlimiting embodiment, the first double-stranded circular DNA vector may comprise the protelomerase gene sequence in the region that is different from the region where the nucleic acid sequence of interest is placed between the pair of protelomerase recognition sequences (that is, in the same region as the at least one endonuclease recognition sequence between the pair of protelomerase recognition sequences).

Nonlimiting Embodiment (ii): The first double-stranded circular DNA vector comprises the endonuclease gene sequence, and the second double-stranded circular DNA vector comprises the protelomerase gene sequence. In the present embodiment, the first double-stranded circular DNA vector may comprise the endonuclease gene sequence in the region that is different from the region where the nucleic acid sequence of interest is placed between the pair of protelomerase recognition sequences (that is, in the same region as the at least one endonuclease recognition sequence between the pair of protelomerase recognition sequences).

Nonlimiting Embodiment (iii): The second double-stranded circular DNA vector comprises the protelomerase gene sequence and the endonuclease gene sequence.

Nonlimiting Embodiment (iv): The transformed cell further comprises the third double-stranded circular DNA vector; the second double-stranded circular DNA vector comprises the protelomerase gene sequence; and the third double-stranded circular DNA vector comprises the endonuclease gene sequence and an endonuclease recognition sequence.

In the transformed cell of the present aspect, in the first double-stranded circular DNA vector, an endonuclease recognition sequence is placed in the region that is different from the region where the nucleic acid sequence of interest is placed between the pair of protelomerase recognition sequences. The endonuclease recognition sequence may be placed adjacent to one of the protelomerase recognition sequences, or a spacer sequence may be placed between the endonuclease recognition sequence and the protelomerase recognition sequence.

In the present description, “spacer sequence” is a sequence that may be inserted between the endonuclease recognition sequence and the protelomerase recognition sequence in the first double-stranded circular DNA vector such that the protelomerase or an active fragment thereof can recognize the protelomerase recognition sequence, and such that the endonuclease or an active fragment thereof can recognize the endonuclease recognition sequence. The base length of the spacer sequence is not limited as long as it is not less than 1 base. The base length is, for example, not less than 1 base, not less than 2 bases, not less than 3 bases, not less than 4 bases, not less than 5 bases, not less than 6 bases, not less than 7 bases, not less than 8 bases, not less than 9 bases, not less than 10 bases, not less than 11 bases, not less than 12 bases, not less than 13 bases, not less than 14 bases, not less than 15 bases, not less than 20 bases, not less than 25 bases, not less than 30 bases, not less than 35 bases, not less than 40 bases, not less than 45 bases, not less than 50 bases, not less than 55 bases, not less than 60 bases, not less than 65 bases, not less than 70 bases, not less than 75 bases, not less than 80 bases, not less than 85 bases, not less than 90 bases, not less than 95 bases, not less than 100 bases, not less than 120 bases, not less than 150 bases, or not less than 200 bases, and/or is not more than 1000 bases, not more than 900 bases, not more than 800 bases, not more than 700 bases, not more than 600 bases, not more than 500 bases, not more than 400 bases, not more than 300 bases, or not more than 250 bases. The base length is, for example, 1 to 1000 bases, 1 to 500 bases, 1 to 400 bases, 1 to 300 bases, 1 to 250 bases, 1 to 200 bases, 1 to 150 bases, 1 to 100 bases, 1 to 70 bases, 1 to 50 bases, 1 to 40 bases, 1 to 30 bases, 1 to 20 bases, 1 to 10 bases, 2 to 10 bases, 3 to 10 bases, 4 to 9 bases, 5 to 9 bases, or 6 to 8 bases.

In the transformed cell of the present aspect, the first double-stranded circular DNA vector may comprise another sequence in addition to the pair of protelomerase recognition sequences, the at least one endonuclease recognition sequence, and the nucleic acid sequence of interest. Further, in the transformed cell of the present aspect, the second double-stranded circular DNA vector may comprise another sequence in addition to the protelomerase gene sequence and/or the endonuclease gene sequence, and the at least one endonuclease recognition sequence. The other sequence is not particularly limited, and may be appropriately selected depending on the purpose. Examples of the other sequence include elements essential for common plasmid vectors, for example, sequences necessary for the maintenance and the replication of the vector in a cell, such as a replication initiation region and/or a gene encoding an antibiotic-resistance protein, and also include sequences encoding an activator protein or a repressor protein such as the LacI gene or the AraC gene, a cloning site, and an overlap region for using the In-Fusion cloning system (Clontech), the Gibson Assembly system (New England Biolabs), or the like. Examples of the gene encoding an antibiotic-resistance protein include the β-lactamase gene, which provides ampicillin resistance (which may be referred to as “ampR gene”), the aminoglycoside 3′-phosphotransferase gene, which provides kanamycin resistance (which may be referred to as “kanR gene”), the tetracycline efflux transporter gene, which provides tetracycline resistance, and the chloramphenicol acetyltransferase (CAT) gene, which provides chloramphenicol resistance.

In the present aspect, the other sequence may be placed in either one or both of the region that is the same as the region where the endonuclease recognition sequence is placed between the pair of protelomerase recognition sequences, or the region that is the same as the region where the nucleic acid sequence of interest is placed between the pair of protelomerase recognition sequences. The other sequence is more particularly placed in the region that is the same as the region where the endonuclease recognition sequence is placed between the pair of protelomerase recognition sequences. For example, all elements other than the nucleic acid sequence of interest may be placed in the same region as the endonuclease recognition sequence.

In one or more embodiments, the first double-stranded circular DNA vector and the second double-stranded circular DNA vector comprise antibiotic-resistance genes each of which provides resistance to an antibiotic. The types of the antibiotic-resistance genes may be those which provide resistance to the same antibiotic, or may be those which provide resistance to different antibiotics, between the first double-stranded circular DNA vector and the second double-stranded circular DNA vector. From the viewpoint of maintaining both double-stranded circular DNA vectors, the antibiotic-resistance genes particularly provide resistance to different antibiotics. Similarly, in cases where the transformed cell of the present aspect comprises the first, second, and third double-stranded circular DNA vectors, the types of the antibiotic-resistance genes may be those which provide resistance to the same antibiotic, or may be those which provide resistance to different antibiotics. From the viewpoint of maintaining the three double-stranded circular DNA vectors, the antibiotic-resistance genes particularly provide resistance to three different antibiotics.

In one or more embodiments, the first double-stranded circular DNA vector and the second double-stranded circular DNA vector comprise replication initiation regions. The types of the replication initiation regions may be those which cause replication by an identical replication mechanism between the first double-stranded circular DNA vector and the second double-stranded circular DNA vector (for example, replication initiation regions consisting of an identical base sequence), or may be those which cause replication by different replication mechanisms (for example, replication initiation regions consisting of different base sequences). Similarly, in cases where the transformed cell of the present aspect comprises the first, second, and third double-stranded circular DNA vectors, the types of the replication initiation regions may be those which cause replication by an identical replication mechanism (for example, replication initiation regions consisting of an identical base sequence), or may be those which cause replication by different replication mechanisms (for example, replication initiation regions consisting of different base sequences).

In the present description, “replication initiation region” refers to a sequence region having an ability to cause DNA replication initiation reaction. Examples of the replication initiation region include a sequence region, in a double-stranded circular DNA vector such as plasmid DNA, that enables replication of the DNA. In the present description, a replication initiation region may also be referred to as “replication origin sequence”. However, since “replication origin” or “origin of replication” (also called “ori”) means the site at which DNA replication starts, the term “replication initiation region” is particularly used over the term “replication origin sequence” in the present description.

Whether replication initiation regions cause replication by an identical replication mechanism or cause replication by different replication mechanisms can be determined by those skilled in the art based on, for example, sequence identity to a known replication initiation region in the host cell. For example, in Escherichia coli, a ColE1-type replication mechanism (for example, a pUC plasmid and a pET-based plasmid), a p15A-type replication mechanism (for example, a pACYC-based plasmid), a pSC101-type replication mechanism (for example, a pSC101-based plasmid), and the like are known, and replication initiation regions that cause replication by these replication mechanisms are known. Plasmids having an identical or similar replication mechanism, such as a pUC plasmid and a pET-based plasmid, are known to be unable to coexist stably over many generations in the same host unless there is a selection pressure. This is known as “incompatibility”. In contrast, the fact that plasmids having different replication mechanisms can coexist in the same host is referred to as “compatibility”.

The method for producing the first and second double-stranded circular DNA vectors is not particularly limited, and may be appropriately selected depending on the purpose. Examples of the method include those using the total synthesis method, the PCR method, and methods using the In-Fusion cloning system (Clontech), the Gibson Assembly system (New England Biolabs), or the like. For example, each vector can be produced based on a commercially available or known vector, and can be produced based on a pUC vector, a pET vector, a pGEM vector, or the like. In particular, the vector is designed based on a plasmid comprising a pUC-based replication initiation region, such as a pUC19 vector, from the viewpoint of achieving high DNA replication efficiency. The first double-stranded circular DNA vector can also be produced by adding the pair of protelomerase recognition sequences, at least one endonuclease recognition sequence, and the nucleic acid sequence of interest to these commercially available vectors, and the second double-stranded circular DNA vector can also be produced by adding the protelomerase gene sequence and/or the endonuclease gene sequence, and at least one endonuclease recognition sequence to these commercially available vectors.

In the present aspect, the type of transformed cell is not limited. The transformed cell is not limited as long as a double-stranded circular DNA vector can be introduced thereto, and as long as the double-stranded circular DNA vector can be retained and/or replicated therein. Examples of the transformed cell include an archaeal cell, a bacterial cell, or a eukaryotic cell. The bacterium may be Escherichia, a lactic acid bacterium, or Bacillus (for example, Bacillus subtilis). Examples of the eukaryotic cell include a fungal cell (for example, a yeast cell), an algal cell, a plant cell, a protozoan cell, an insect cell, a nematode cell, a fish cell, an avian cell (for example, a chicken cell), and a mammalian cell (for example, a mouse cell, a chimpanzee cell, and a human cell). Among these, a cell capable of replicating a double-stranded circular DNA vector is particularly preferred.

Examples of the Escherichia include Escherichia coli. More specifically, the Escherichia is, for example, the Escherichia coli K-12 strain or the Escherichia coli B strain.

A commercially available Escherichia coli strain, or an Escherichia coli strain from a bioresource center may also be used. For example, the JM109 strain, DH5 strain, DH5a strain, DH10B strain, NEB10ß strain, HST08 strain, HST16CR strain, HB101 strain, W3110 strain, MG1655 strain, BL21 strain, BL21 DE3 strain, or the like may be used. These strains are available from New England Biolabs, Takara Bio Inc., Thermo Fisher Scientific, Inc., Toyobo Co. Ltd., ATCC (American Type Culture Collection), NBRP (National Bio Resource Project), and the like.

Further, in one or more embodiments of the present invention, strains derived from the above-described Escherichia coli strains may also be used. Examples of such strains include the JW3973 strain (available from NBRP), which is auxotrophic for methionine, the JW2806 strain (available from NBRP), which is auxotrophic for leucine, the JW3582 strain (available from NBRP), which is auxotrophic for cysteine, and the ME5305 strain (available from NBRP), which is auxotrophic for thiamine and histidine.

In one or more embodiments, the transformed cell of the present aspect expresses an exonuclease. Here, the exonuclease is not particularly limited as long as DNA cleaved by an endonuclease or an active fragment thereof can be degraded therewith from the cleaved end. The exonuclease may be, for example, an endogenous exonuclease.

### 1-4. Effects

When expression of the protelomerase or an active fragment thereof is induced in the transformed cell of the present aspect, the protelomerase recognition sequences in the first double-stranded circular DNA vector are recognized, and the cleaved ends are rejoined to generate a linear covalently closed DNA of interest comprising the nucleic acid sequence of interest, and to simultaneously generate, as a by-product, a linear covalently closed DNA that does not comprise the nucleic acid sequence of interest. The linear covalently closed DNA that does not comprise the nucleic acid sequence of interest, and the second double-stranded circular DNA vector, are cleaved by the endonuclease or an active fragment thereof through recognition of the endonuclease recognition sequences. As a result, the linear covalently closed DNA that does not comprise the nucleic acid sequence of interest, and the second double-stranded circular DNA vector, are degraded by, for example, endogenous exonuclease activity, so that the linear covalently closed DNA of interest can be efficiently obtained.

In general, as the size of a double-stranded circular DNA vector increases, the copy number of the vector retained in a host decreases. Therefore, in cases where one double-stranded circular DNA vector comprises all of the elements required to generate a linear covalently closed DNA in a cell, the amount of the linear covalently closed DNA produced is considered to decrease when a large-sized nucleic acid sequence of interest is carried on the vector. In contrast, in the transformed cell of the present aspect, the amount of sequence other than the nucleic acid sequence of interest is small in the first double-stranded circular DNA vector, so that even a large-sized linear covalently closed DNA can be produced in a large amount. Further, since the transformed cell of the present aspect comprises two or more double-stranded circular DNA vectors, a high degree of freedom is available for making the vectors carry other sequences. For example, replication initiation regions that cause replication by different replication mechanisms may be introduced into the first and/or second double-stranded circular DNA vector(s).

It was unexpected, from the viewpoint of incompatibility, that a linear covalently closed DNA can be efficiently produced by the transformed cell of the present aspect even in cases where the first double-stranded circular DNA vector and the second double-stranded circular DNA vector comprise replication initiation regions that cause replication by an identical replication mechanism.

### 2. Composition for Transformation

### 2-1. Overview

A second aspect of one or more embodiments of the present invention is a composition for transformation. The composition for transformation of the present aspect comprises at least the first double-stranded circular DNA vector and the second double-stranded circular DNA vector described in the first aspect. The transformed cell of the first aspect can be produced by transforming a host cell with the composition for transformation of the present aspect.

### 2-2. Configuration

The composition for transformation of the present aspect comprises, as essential components, at least the first double-stranded circular DNA vector and the second double-stranded circular DNA vector described in the first aspect. Since the description of the first aspect also applies to the first and second double-stranded circular DNA vectors, detailed description of these vectors is omitted herein.

The composition for transformation of one or more embodiments of the present invention may comprise, as an optional component, the third double-stranded circular DNA vector described in the first aspect. Since the description of the first aspect also applies to the third double-stranded circular DNA vector, detailed description of this vector is omitted herein.

The composition for transformation of the present aspect may comprise, as optional components, a solvent and/or an additive agent. The solvent may be, for example, either water or an aqueous solution, or an organic solvent. Examples of the aqueous solution include physiological saline, phosphate buffer, sodium acetate buffer, and Tris buffer. These are preferably sterilized. Examples of the additive agent include a chelating agent, a pH-adjusting agent, a suspending agent, a surface-activating agent, a stabilizing agent, an excipient, a preserving agent, a diluting agent, a tonicity-adjusting agent, a buffering agent, and a solubilizing agent. A chelating agent such as EDTA or EGTA is especially preferred since such an agent can inactivate a trace amount of nuclease in cases where the composition is contaminated therewith.

### 3. Kit for Transformation

### 3-1. Overview

A third aspect of one or more embodiments of the present invention is a kit for transformation. The kit for transformation of the present aspect comprises at least the first double-stranded circular DNA vector and the second double-stranded circular DNA vector described in the first aspect. The transformed cell of the first aspect can be produced by using the kit for transformation of the present aspect.

### 3-2. Configuration

The kit for transformation of the present aspect comprises, as essential components, at least the first double-stranded circular DNA vector and the second double-stranded circular DNA vector described in the first aspect. Since the description of the first aspect also applies to the first and second double-stranded circular DNA vectors, detailed description of these vectors is omitted herein.

In one or more embodiments, the nucleic acid sequence of interest in the first double-stranded circular DNA vector comprised in the kit for transformation of the present aspect may be a nuclease recognition sequence for introducing a nucleic acid sequence of interest. This nuclease recognition sequence may be a multi-cloning site capable of introducing various nucleic acid sequences.

The kit for transformation of the present aspect may comprise, as an optional component, the third double-stranded circular DNA vector described in the first aspect. Since the description of the first aspect also applies to the third double-stranded circular DNA vector, its detailed description is omitted herein.

In one or more embodiments of the kit for transformation of the present aspect, the first, second, and third double-stranded circular DNA vectors are provided in separate sealed containers.

The kit for transformation of the present aspect may comprise, as optional components, other reagents necessary for transformation, such as a buffer, a transformation reagent, an antibiotic used for selection of a cell after transformation, and/or an instruction manual describing a transformation method.

### 4. Method for Producing Linear Covalently Closed DNA

### 4-1. Overview

A fourth aspect of one or more embodiments of the present invention is a method for producing a linear covalently closed DNA. The production method of the present aspect comprises, as essential steps, a culturing step, an expression-inducing step, and a DNA-extracting step, and comprises, as optional steps, an isolating step and/or an introducing step. According to the production method of the present aspect, a linear covalently closed DNA can be produced conveniently and efficiently.

### 4-2. Method

The steps in the method for producing a linear covalently closed DNA of the present aspect are specifically described below.

### (Introducing Step)

The “introducing step” is a step of introducing a first and/or second double-stranded circular DNA vector(s) into a host cell to obtain a transformed cell comprising the first double-stranded circular DNA vector and the second double-stranded circular DNA vector. In one or more embodiments, the present step introduces the composition for transformation of the second aspect into a host cell. In other embodiments, the present step introduces the first double-stranded circular DNA vector described in the first aspect into a host cell that has been transformed with the second double-stranded circular DNA vector, or with the second and third double-stranded circular DNA vectors, described in the first aspect. In still other embodiments, the present step introduces the second double-stranded circular DNA vector, or the second and third double-stranded circular DNA vectors, described in the first aspect into a host cell that has been transformed with the first double-stranded circular DNA vector described in the first aspect.

The method for introducing the first and/or second double-stranded circular DNA vector(s) into a host cell in the present step is not particularly limited. For example, a gene transfer method (transformation method) known in the art described in, for example, Green & Sambrook, 2012, Molecular Cloning: A Laboratory Manual Fourth Ed., Cold Spring Harbor Laboratory Press, Cold Spring Harbor, New York may be used. Specific examples of the method include the heat shock method, the lipofection method, the electroporation method, the microinjection method, the calcium phosphate method, the DEAE-dextran method, introduction using a cationic lipid, introduction using a cationic polymer (such as polyethyleneimine (PEI)), introduction using a nanoparticle, introduction using a virus, and particle bombardment.

In the present step, the host cell into which the first and/or second double-stranded circular DNA vector(s) has/have been introduced can be appropriately selected in a medium comprising an antibiotic based on a gene encoding an antibiotic-resistance protein in the first and/or second double-stranded circular DNA vector(s).

### (Culturing Step)

The “culturing step” is a step of culturing a transformed cell into which the first and second double-stranded circular DNA vectors have been introduced. The present step aims to proliferate the transformed cell before the later-described expression-inducing step, thereby increasing the intracellular copy number of the double-stranded circular DNA vectors before proceeding to the later-described expression-inducing step.

The method for culturing the transformed cell in the present step is not particularly limited, and can be appropriately selected according to the type of the transformed cell. For example, a culturing method known in the art described in, for example, Green & Sambrook, 2012, Molecular Cloning: A Laboratory Manual Fourth Ed., Cold Spring Harbor Laboratory Press, Cold Spring Harbor, New York may be used.

The medium for culturing the transformed cell in the present step may be either a liquid medium or a solid medium. The medium may be a medium comprising one or more components selected from, for example, enzymatic degradation products of protein, such as peptone and tryptone; organism-derived extracts such as potato dextrose and yeast extract; amino acids such as glutamic acid or a salt thereof, sugars such as glucose, glycerol, and sucrose; and inorganic salts such as sodium chloride, magnesium chloride, and potassium dihydrogen phosphate. Specific examples of the medium and its composition include LB medium (tryptone, yeast extract, sodium chloride), YPG medium (yeast extract, peptone, glucose), PD medium (potato dextrose), and TB medium (tryptone, yeast extract, dipotassium hydrogen phosphate, potassium dihydrogen phosphate).

In cases where the first and second double-stranded circular DNA vectors comprise genes encoding antibiotic-resistance proteins, the present step may be carried out in the presence of two types of antibiotics corresponding to the genes encoding antibiotic-resistance proteins in the first and second double-stranded circular DNA vectors. Further, in cases where the transformed cell comprises a third double-stranded circular DNA vector comprising a gene encoding an antibiotic-resistance protein, culturing in the present step may be performed in the presence of three types of antibiotics. Examples of the antibiotic include ampicillin, carbenicillin, kanamycin, tetracycline, or chloramphenicol.

The culturing conditions in the present step may be appropriately selected according to the type of the transformed cell. For example, culturing in the present step may be performed at 20 to 42° C., 25 to 40° C., 30 to 38° C., or 35 to 37° C.

The culturing time in the present step is not limited as long as a sufficient amount of the double-stranded circular DNA vectors can be obtained. For example, the culturing time may be not less than 1 hour, not less than 2 hours, not less than 4 hours, not less than 12 hours, not less than 24 hours, not less than 2 days, not less than 3 days, or not less than 1 week.

The pH of the medium used in the present step is not particularly limited as long as the transformed cell can grow. For example, the pH may be not less than 3, not less than 4, not less than 5, not less than 6, not less than 7, not less than 7.5, not less than 8, not less than 8.5, not less than 9, or not less than 9.5, and/or not more than 11, not more than 10.5, not more than 10, not more than 9.5, not more than 9, not more than 8.5, not more than 8, or not more than 7.5. The pH may also be, for example, 3 to 11, 4 to 10, 5 to 9, 7 to 9, 7.5 to 10.5, 8 to 10, or 8.5 to 9.5

### (Expression-Inducing Step)

In the present aspect, the “expression-inducing step” is a step of inducing the expression of the endonuclease or an active fragment thereof, and the protelomerase or an active fragment thereof, in the transformed cell after the culturing step.

The method for inducing the expression of the endonuclease or an active fragment thereof and the protelomerase or an active fragment thereof in the present step is not particularly limited, and may be appropriately selected according to the type of the promoter capable of controlling expression.

In one or more embodiments, in the present step, an expression-inducing agent is added to the medium after the culturing step. For example, in cases of an arabinose-inducible promoter, an IPTG-inducible promoter, or a rhamnose-inducible promoter, examples of the method include a method in which arabinose, IPTG, or rhamnose is added as an expression-inducing agent to the medium comprising the transformed cell after the culturing step.

The length of time for which the transformed cell is cultured in the presence of the expression-inducing agent in the present step is not particularly limited as long as expression of the endonuclease or an active fragment thereof and the protelomerase or an active fragment thereof is sufficiently induced. For example, the length of time may be not less than 1 minute, not less than 5 minutes, not less than 10 minutes, not less than 20 minutes, not less than 30 minutes, not less than 1 hour, not less than 2 hours, not less than 3 hours, not less than 6 hours, not less than 12 hours, or not less than 24 hours.

The pH of the medium used in the present step may be in a range in which the endonuclease or an active fragment thereof and/or the protelomerase or an active fragment thereof can function in a cell. The pH may be, for example, 3 to 11, 4 to 10, 5 to 9, or 7 to 9. For example, a pH range between the optimum pH of the protelomerase and the optimum pH of the endonuclease may be used.

In the present step, the temperature at which the transformed cell is cultured in the presence of the expression-inducing agent may be, for example, not less than 20° C., not less than 25° C., not less than 28° C., not less than 30° C., or not less than 31° C., and/or not more than 42° C., not more than 40° C., not more than 39° C., not more than 38° C., not more than 37° C., not more than 36° C., not more than 35° C., not more than 34° C., not more than 33° C., not more than 32° C., or not more than 31° C. The temperature may also be, for example, 26 to 38° C., 27 to 37° C., 28 to 36° C., 29 to 35° C., 30 to 34° C., 30 to 33° C., 30 to 32° C., or 30 to 31° C. For example, a temperature range between the optimum temperature of the protelomerase and the optimum temperature of the endonuclease may be used.

In further embodiments, in the present step, the transformed cell may be cultured under a plurality of temperature conditions in the presence of the expression-inducing agent. The plurality of temperature conditions may be selected from the optimal temperature ranges of the enzyme activities of the protelomerase and the endonuclease, respectively. The optimal temperature of the enzyme activity of the protelomerase may be, for example, 26 to 37° C., 27 to 35° C., 28 to 33° C., or 29 to 31° C. (such as 30° C.). The optimal temperature of the enzyme activity of the homing endonuclease may be, for example, 30 to 42° C., 32 to 40° C., 34 to 39° C., or 36 to 38° C. (such as 37° C.). In one or more embodiments, the culturing time under each temperature condition may be not less than 10 minutes, not less than 20 minutes, not less than 30 minutes, not less than 1 hour, not less than 2 hours, not less than 3 hours, not less than 6 hours, not less than 12 hours, or not less than 24 hours.

In other embodiments, in cases where the promoter capable of controlling expression is a heat-inducible promoter or a cold-inducible promoter, heat shock or cold shock may be applied. The conditions of the heat shock or the cold shock are not particularly limited as long as the temperature and the length of time allow sufficient induction of the expression of the endonuclease or an active fragment thereof and the protelomerase or an active fragment thereof. For example, the heat shock may be performed at 36 to 45° C., 37 to 44° C., 38 to 43° C., 39 to 42° C., or 40 to 41° C. for not less than 10 seconds, not less than 20 seconds, not less than 30 seconds, not less than 40 seconds, not less than 50 seconds, not less than 1 minute, not less than 5 minutes, not less than 10 minutes, not less than 20 minutes, not less than 30 minutes, or not less than 1 hour, and/or not more than 24 hours, not more than 12 hours, not more than 6 hours, not more than 3 hours, or not more than 2 hours. The heat shock may be performed, for example, for 10 seconds to 24 hours, 20 seconds to 12 hours, 25 seconds to 6 hours, 30 seconds to 3 hours, 40 seconds to 2 hours, 50 seconds to 1 hour, 1 minute to 30 minutes, or 2 minutes to 5 minutes.

By the present step, the endonuclease or an active fragment thereof and the protelomerase or an active fragment thereof are expressed in the transformed cell, resulting in generation of a linear covalently closed DNA of interest comprising the nucleic acid sequence of interest and, as a by-product, a linear covalently closed DNA that does not comprise the nucleic acid sequence of interest. Further, the linear covalently closed DNA not comprising the nucleic acid sequence of interest; and the second double-stranded circular DNA vector comprising the protelomerase gene sequence and/or the endonuclease gene sequence, and the endonuclease recognition sequence; are cleaved, and then degraded by exonuclease activity.

### (DNA-Extracting Step)

The “DNA-extracting step” is a step of extracting DNA from the transformed cell after the expression-inducing step.

The method used for extracting DNA in the present step may be a conventional method known in the art. For example, the extraction may be performed according to the method described in Green & Sambrook, 2012, Molecular Cloning: A Laboratory Manual Fourth Ed., Cold Spring Harbor Laboratory Press, Cold Spring Harbor, New York. Alternatively, the extraction may be performed using a commercially available DNA extraction kit.

By the present step, a linear covalently closed DNA comprising the nucleic acid sequence of interest is extracted.

### (Isolating Step)

The “isolation step” is a step of isolating a linear covalently closed DNA after the DNA-extracting step. The present step aims to remove DNA components other than the linear covalently closed DNA comprising the nucleic acid sequence of interest, such as a genomic DNA fragment and an uncut DNA fragment extracted in the DNA-extracting step.

The method for isolating the linear covalently closed DNA in the present step is not particularly limited, and may be appropriately selected. Examples of the method include a method in which purification is performed using a commercially available kit, and a method in which isolation is performed using gel extraction, cation-exchange chromatography, anion-exchange chromatography, size-exclusion chromatography, a filter, or an ultrafiltration membrane.

### 4-3. Effects

According to the production method of the present aspect, a linear covalently closed DNA can be produced conveniently and efficiently. Based on the production method of the present aspect, mass production of a linear covalently closed DNA can be achieved with a small number of steps.

In one or more embodiments in which the introducing step of the production method of the present aspect is carried out by introducing the first and second double-stranded circular DNA vectors into the host cell in a manner in which the first double-stranded circular DNA vector is introduced into a host cell that has been transformed with the second double-stranded circular DNA vector, the host cell comprising the second double-stranded circular DNA vector may be first produced, and then the common host cell may be transformed with first double-stranded circular DNA vectors comprising different nucleic acid sequences of interest. By this, a plurality of transformed cells can be efficiently produced. Similarly, in one or more embodiments in which the first, second, and third double-stranded circular DNA vectors are introduced into the host cell in a manner in which the first double-stranded circular DNA vector is introduced into a host cell that has been transformed with the second and third double-stranded circular DNA vectors, the host cell comprising the second and third double-stranded circular DNA vectors may be first produced, and then the common host cell may be transformed with first double-stranded circular DNA vectors comprising different nucleic acid sequences of interest. By this, a plurality of transformed cells can be efficiently produced.

### EXAMPLES

One or more embodiments of the present invention are described below specifically by way of Examples. These Examples are for description of one or more embodiments of the present invention, and do not limit the scope of one or more embodiments of the present invention.

### Comparative Example 1: Construction of Comparative Example Vector (T6) for Producing Linear Covalently Closed DNA

### Object

A Comparative Example vector (T6) for producing a linear covalently closed DNA is produced.

### Methods and Results

In the following Examples, nucleic acid manipulation based on recombinant DNA techniques was performed with reference to the following two documents: Molecular Cloning 2nd Edition (Cold Spring Harbor Laboratory Press, 1989), and Current Protocols in Molecular Biology (Greene Publishing Associates and Wiley-Interscience).

For PCR in the following Examples, Prime STAR MAX DNA Polymerase (manufactured by Takara Bio Inc.) and the like were used, and the reaction conditions followed the methods described in the attached manual.

### (1) Preparation of Nucleic Acid Fragment

A nucleic acid fragment to be used for construction of a Comparative Example vector (T6) for producing a linear covalently closed DNA was produced as follows.

### (a) Nucleic Acid Encoding Fusion Polypeptide (SEQ ID NO: 1)

A nucleic acid (SEQ ID NO: 1) encoding a fusion polypeptide comprising, in the order from the N-terminal side, TelN protelomerase (a protelomerase derived from the Escherichia virus N15), a linker sequence (the amino acid sequence encoded by the base sequence of SEQ ID NO: 34), and I-SceI homing endonuclease (a homing endonuclease derived from Saccharomyces cerevisiae) that are linked together was produced by PCR using primer 1 (forward, SEQ ID NO: 14) and primer 4 (reverse, SEQ ID NO: 17).

### (b) Nucleic Acid Encoding TelN Protelomerase (SEQ ID NO: 2)

A nucleic acid encoding TelN protelomerase (SEQ ID NO: 2) was produced by PCR using the nucleic acid encoding the above-described fusion polypeptide (SEQ ID NO: 1) as a template, and using primer 1 (forward, SEQ ID NO: 14) and primer 2 (reverse, SEQ ID NO: 15).

(c) Pair of Protelomerase Recognition Sequences (telRL Sequences, SEQ ID NO: 3)

A nucleic acid comprising a pair of protelomerase recognition sequences (telRL sequences, SEQ ID NO: 3) was produced by total synthesis.

### (d) Nucleic Acid Encoding I-SceI Homing Endonuclease (SEQ ID NO: 4)

A nucleic acid encoding I-SceI homing endonuclease derived from Saccharomyces cerevisiae (SEQ ID NO: 4) was produced by PCR using the nucleic acid encoding the above-described fusion polypeptide (SEQ ID NO: 1) as a template, and using primer 3 (forward, SEQ ID NO: 16) and primer 4 (reverse, SEQ ID NO: 17).

(e) Sequence Comprising telRL Sequence and I-SceI Recognition Sequence (SEQ ID NO: 8)

A sequence comprising a telRL sequence and an I-SceI recognition sequence (SEQ ID NO: 8) was produced by total synthesis. SEQ ID NO: 8 comprises T6 as a spacer sequence between the telRL sequence and the I-SceI recognition sequence.

### (f) AraC Gene Sequence Under Control of Promoter (SEQ ID NO: 11)

An AraC gene sequence under the control of a promoter (SEQ ID NO: 11) was produced by PCR using primer 5 (forward, SEQ ID NO: 18) and primer 6 (reverse, SEQ ID NO: 19).

### (g) Arabinose-Inducible Promoter Sequence (SEQ ID NO: 12)

An arabinose-inducible promoter sequence (SEQ ID NO: 12) was produced by total synthesis.

### (h) Arabinose-Inducible Promoter Sequence Comprising Regulatory Sequence (SEQ ID NO: 13)

An arabinose-inducible promoter sequence comprising a regulatory sequence (SEQ ID NO: 13) was produced by PCR using primer 7 (forward, SEQ ID NO: 20) and primer 8 (reverse, SEQ ID NO: 21).

### (2) Production of Comparative Example Vector (T6)

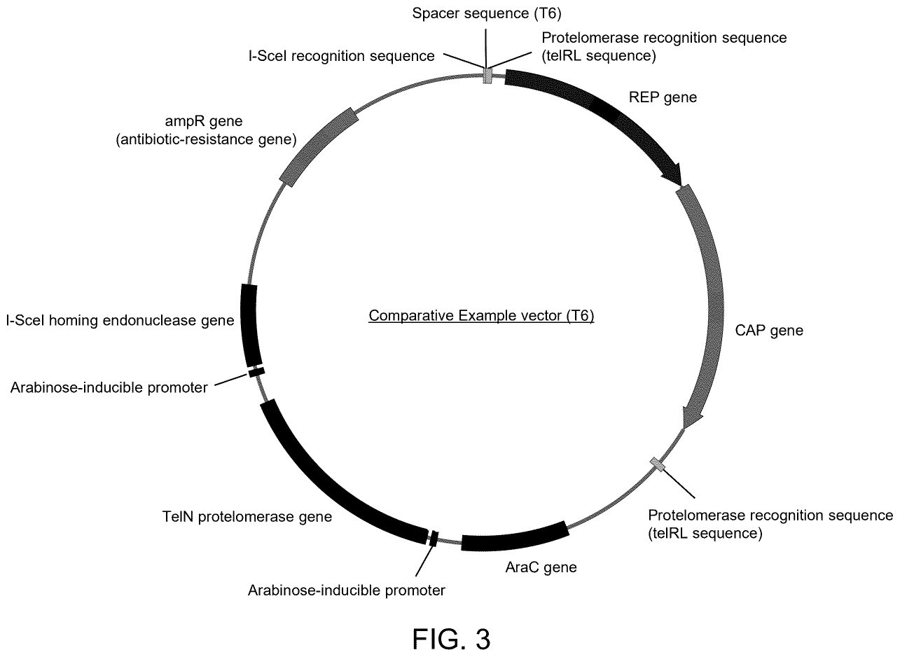

As a vector to be used as a Comparative Example for producing a linear covalently closed DNA, a vector comprising a TelN protelomerase gene and an I-SceI homing endonuclease gene (hereinafter referred to as “Comparative Example vector (T6)”) was produced (FIG. 3).

The method for producing the Comparative Example vector (T6) was as follows. First, the telRL sequence of (c) was inserted into the SnaBI site of the pRC2-mi342 vector (manufactured by Takara Bio Inc.). Subsequently, (b), (d), (f), (g), and (h) described above were inserted into the Smal site of this vector. The sequence (e), comprising the telRL sequence and the I-SceI recognition sequence (SEQ ID NO: 8), was inserted into the EcoRV site of the resulting vector, to produce a Comparative Example vector (T6) (FIG. 3).

The Comparative Example vector (T6) is designed such that the I-SceI homing endonuclease and the TelN protelomerase are expressed under the control of the arabinose-inducible promoter. Further, a Rep gene and a Cap gene are placed between the pair of protelomerase recognition sequences (telRL sequences) (FIG. 3).

### Example 1: Construction of Two Vectors for Producing Linear Covalently Closed DNA

### Object

A control vector (T6) and an I-SceI vector are produced as two vectors for producing a linear covalently closed DNA.

### Methods and Results

### (1) Preparation of Nucleic Acid Fragment

As a nucleic acid fragment used for construction of a vector, a nucleic acid (SEQ ID NO: 35) encoding aminoglycoside 3′-phosphotransferase, which provides kanamycin resistance, was produced by total synthesis.

### (2) Preparation of Control Vector (T6)

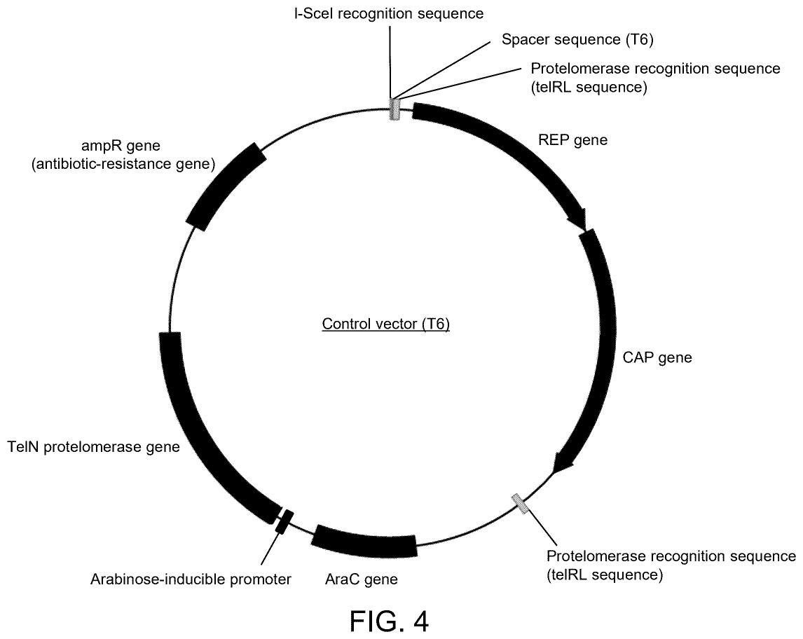

A vector that comprises T6 as a spacer sequence between a telRL sequence and an I-SceI recognition sequence, and that expresses TelN protelomerase under the control of an arabinose-inducible promoter (hereinafter referred to as “control vector (T6)”), was produced (FIG. 4).

The method for producing the control vector (T6) was as follows. First, a nucleic acid fragment was produced by PCR using the Comparative Example vector (T6) produced in Comparative Example 1 as a template, and using primer 9 (forward, SEQ ID NO: 36) and primer 10 (reverse, SEQ ID NO: 37). Subsequently, the resulting nucleic acid fragment was circularized by the Gibson Assembly system (New England Biolabs) using overlap regions at the ends of the obtained nucleic acid fragment, to produce the control vector (T6) (FIG. 4). The control vector (T6) is a vector obtained by removing, from the Comparative Example vector (T6), the I-SceI homing endonuclease gene designed to be expressed under the control of the arabinose-inducible promoter. In the control vector (T6), a Rep gene and a Cap gene are placed between a pair of protelomerase recognition sequences (telRL sequences) (FIG. 4).

### (3) Preparation of I-SceI Vector

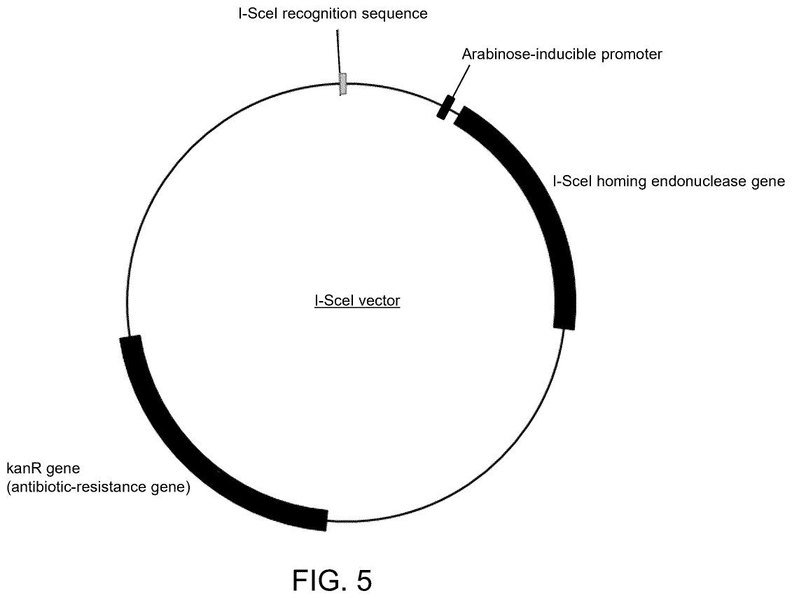

A vector that comprises a nucleic acid encoding aminoglycoside 3′-phosphotransferase for providing kanamycin resistance (SEQ ID NO: 35), and that expresses I-SceI homing endonuclease under the control of an arabinose-inducible promoter (hereinafter referred to as “I-SceI vector”), was produced (FIG. 5).

The method for producing the I-SceI vector was as follows. First, in the Comparative Example vector (T6) produced in Comparative Example 1, the nucleic acid encoding β-lactamase for providing ampicillin resistance was replaced with a nucleic acid (SEQ ID NO: 35) encoding aminoglycoside 3′-phosphotransferase for providing kanamycin resistance. A nucleic acid fragment was produced by PCR using the vector after the replacement as a template, and using primer 7 (forward, SEQ ID NO: 20) and primer 11 (reverse, SEQ ID NO: 38). Subsequently, the resulting nucleic acid fragment was circularized by the Gibson Assembly system (New England Biolabs) using overlap regions at the ends of the fragment, to produce the I-SceI vector (FIG. 5). The I-SceI vector does not comprise a protelomerase recognition sequence (telRL sequence), but comprises an I-SceI recognition sequence (FIG. 5).

Since the replication initiation region of the control vector (T6) and the replication initiation region of the I-SceI vector consist of the same base sequence, these vectors have the same replication mechanism.

### Example 2: Production of Linear Covalently Closed DNA

### Object

Escherichia coli is transformed with the control vector (T6) and the I-SceI vector produced in Example 1, to produce a linear covalently closed DNA. The amount of the linear covalently closed DNA produced is compared with that in a case where Escherichia coli is transformed with the Comparative Example vector (T6) produced in Comparative Example 1.

### Methods and Results

### (1) Transformation

Escherichia coli was transformed with the control vector (T6) and the I-SceI vector produced in Example 1. Further, as a control group, Escherichia coli transformed with only the control vector (T6) was produced.

After mixing 25 μL of a competent cell solution of the Escherichia coli DH10B strain with a solution comprising each vector, the resulting mixture was left to stand on ice for 30 minutes. Thereafter, the mixture was heat-treated at 42° C. for 45 seconds, and then left to stand on ice for 2 minutes. Subsequently, 225 μL of SOC medium was added. In the case where only the control vector (T6) was introduced, Escherichia coli was applied to LB agar medium (1% tryptone, 0.5% dried yeast extract, 1% sodium chloride, 0.005% carbenicillin disodium (manufactured by Nacalai Tesque, Inc.)). In the case where the control vector (T6) and the I-SceI vector were introduced, Escherichia coli was plated on LB agar medium (1% tryptone, 0.5% dried yeast extract, 1% sodium chloride, 0.005% carbenicillin disodium (manufactured by Nacalai Tesque, Inc.), 0.005% kanamycin sulfate (manufactured by FUJIFILM Wako Pure Chemical Corporation)). Strains that grew in static culture at 37° C. for 1 day were selected to obtain Escherichia coli into which the vectors had been introduced.

### (2) Culture

The Escherichia coli into which the Comparative Example vector (T6) obtained in Comparative Example 1 had been introduced was inoculated into 2 mL of Plusgrow II medium (4% Plusgrow II (manufactured by Nacalai Tesque, Inc.), 0.005% carbenicillin disodium), and shake culture was performed at 37° C. for 6 hours, to obtain a preculture liquid. For subculture, 200 μL of the preculture liquid was inoculated into 100 mL of Plusgrow II medium (4% Plusgrow II, 0.005% carbenicillin disodium) in a flask, and shake culture was performed at 37° C. for 16 hours.

The Escherichia coli into which only the control vector (T6) had been introduced, obtained as described above, was inoculated into 2 mL of Plusgrow II medium (4% Plusgrow II, 0.005% carbenicillin disodium), and shake culture was performed at 37° C. for 6 hours, to obtain a preculture liquid. For subculture, 200 μL of the preculture liquid was inoculated into 100 mL of Plusgrow II medium (4% Plusgrow II, 0.005% carbenicillin disodium) in a flask, and shake culture was performed at 37° C. for 16 hours.

The Escherichia coli into which the control vector (T6) and the I-SceI vector had been introduced, obtained as described above, was inoculated into 2 mL of Plusgrow II medium (4% Plusgrow II, 0.005% carbenicillin disodium, 0.005% kanamycin sulfate), and shake culture was performed at 37° C. for 6 hours, to obtain a preculture liquid. For subculture, 200 μL of the preculture liquid was inoculated into 100 mL of Plusgrow II medium (4% Plusgrow II, 0.005% carbenicillin disodium, 0.005% kanamycin sulfate) in a flask, and shake culture was performed at 37° C. for 16 hours.

### (3) Induction of Expression

To the culture liquid obtained from the Escherichia coli into which the Comparative Example vector (T6) had been introduced, 100 mL of Plusgrow II medium (4% Plusgrow II, 0.005% carbenicillin disodium) at pH 7.0 and 2 mL of an arabinose solution (10% arabinose) were added, followed by performing shake culture at 30° C. or 37° C. for 6 hours. Thereafter, bacterial cells were collected by centrifugation.

To the culture liquid obtained from the Escherichia coli into which only the control vector (T6) had been introduced, 100 mL of Plusgrow II medium (4% Plusgrow II, 0.005% carbenicillin disodium) at pH 7.0 and 2 mL of an arabinose solution (10% arabinose) were added, followed by performing shake culture at 30° C. or 37° C. for 6 hours. Thereafter, bacterial cells were collected by centrifugation.

To the culture liquid obtained from the Escherichia coli into which the control vector (T6) and the I-SceI vector had been introduced, 100 mL of Plusgrow II medium (4% Plusgrow II, 0.005% carbenicillin disodium, 0.005% kanamycin sulfate) at pH 7.0 and 2 mL of an arabinose solution (10% arabinose) were added, followed by performing shake culture at 30° C. or 37° C. for 6 hours. Thereafter, bacterial cells were collected by centrifugation.

### (4) DNA Extraction

DNA was extracted from 1 mL of the collected culture liquid using a QIA prep Spin Miniprep Kit (manufactured by QIAGEN).

### (5) Evaluation of Total DNA Yield

Table 1 below shows the results of calculation of the total yield of DNA obtained from 200 mL of the culture liquid based on measurement using Nanodrop (manufactured by Thermo Fisher Scientific).

TABLE 1
Results of measurement of total DNA yield

| | |
|---|---|
| | Total DNA yield from 200 |
| Vector for | mL of culture liquid (mg) |

| producing linear | Expression-inducing | Expression-inducing |
|---|---|---|
| covalently | culture temperature: | culture temperature: |
| closed DNA | 30° C. | 37° C. |
| Comparative | 2.64 | 2.46 |
| Example vector (T6) | | |
| Control vector (T6) | 4.51 | 3.92 |
| Control vector (T6) | 2.67 | 2.31 |
| and | | |
| I-SceI vector | | |

### (6) Evaluation of Linear Covalently Closed DNA

The linear covalently closed DNA was evaluated by electrophoresis. Specifically, the DNA solution obtained in (4) above, after adjusting the concentration to 2 to 10 ng/μL, was analyzed using a microchip electrophoresis apparatus for DNA/RNA analysis, MultiNA (manufactured by Shimadzu Corporation).

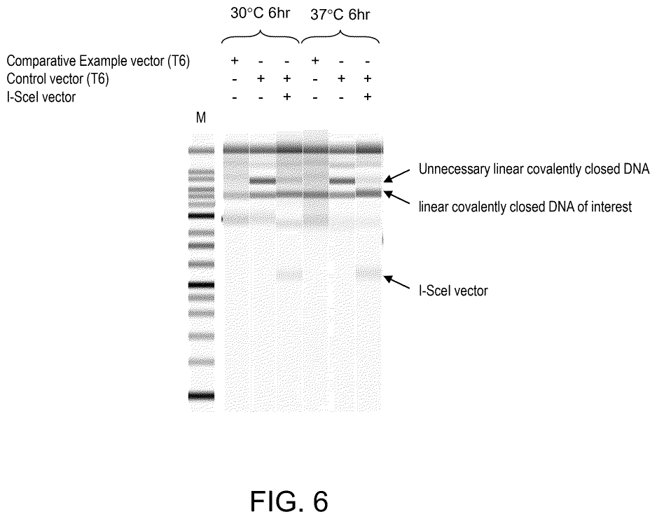

FIG. 6 shows the results of electrophoresis obtained by MultiNA. In FIG. 6, a linear covalently closed DNA comprising a nucleic acid encoding the packaging protein is indicated as “linear covalently closed DNA of interest”, and a linear covalently closed DNA derived from a vector sequence other than the nucleic acid encoding the packaging protein is indicated as “unnecessary linear covalently closed DNA”. In the figure, the left side shows samples subjected to shake culture at an expression-inducing culture temperature of 30° C. for 6 hours, and the right side shows samples subjected to shake culture at an expression-inducing culture temperature of 37° C. for 6 hours.

Further, the residual ratio of the unnecessary linear covalently closed DNA was calculated from the molar concentrations of the linear covalently closed DNA of interest and the unnecessary linear covalently closed DNA. The equation that was used for calculating the residual ratio is shown below.

Residual ratio (%)=(number of moles of unnecessary linear covalently closed DNA)/(number of moles of linear covalently closed DNA of interest)×100

Table 2 below shows the result of calculation of the residual ratio under each condition.

TABLE 2
Residual ratio of unnecessary linear covalently closed DNA

| | Residual ratio of unnecessary | |
|---|---|---|
| | linear covalently closed DNA (%) | |
| Vector for producing | Expression-inducing | Expression-inducing |
| linear covalently | culture temperature: | culture temperature: |
| closed DNA | 30° C. | 37° C. |
| Comparative Example | 41.4 | 28.9 |
| vector (T6) | | |
| Control vector (T6) | 93.5 | 91.4 |
| Control vector (T6) | 33.2 | 9.6 |
| and | | |
| I-SceI vector | | |

According to results shown in FIG. 6 and Table 2, for the control vector (T6), the residual ratios of the unnecessary linear covalently closed DNA were 93.5% (30° C.) and 91.4% (37° C.). In contrast, under the conditions where the control vector (T6) and the I-SceI vector were introduced, the residual ratios of the unnecessary linear covalently closed DNA were significantly reduced (33.2% (30° C.) and 9.6% (37° C.)). These results suggest that the homing endonuclease recognition sequence was cleaved by I-SceI homing nuclease expressed from the I-SceI vector, followed by degradation of the DNA from the cleaved end due to endogenous exonuclease in the Escherichia coli cells.

All publications, patents, and patent applications cited in the present description are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A transformed cell comprising a first double-stranded circular DNA vector and a second double-stranded circular DNA vector, comprising:
a protelomerase gene sequence encoding a protelomerase or an active fragment thereof which is placed under the control of a promoter capable of controlling expression; and
an endonuclease gene sequence encoding an endonuclease or an active fragment thereof which is placed under the control of a promoter capable of controlling expression,
wherein the first double-stranded circular DNA vector comprises:
a pair of protelomerase recognition sequences which are recognized and cleaved by the protelomerase or an active fragment thereof;
a nucleic acid sequence of interest which is placed in a region between the pair of protelomerase recognition sequences; and
at least one endonuclease recognition sequence which is placed in an other region between the pair of protelomerase recognition sequences and which is recognized and cleaved by the endonuclease or an active fragment thereof, and
wherein the second double-stranded circular DNA vector comprises:
one or more of the protelomerase gene sequence and the endonuclease gene sequence; and
at least one endonuclease recognition sequence which is recognized and cleaved by the endonuclease or an active fragment thereof.

2. The transformed cell of claim 1, wherein two or more double-stranded circular DNA vectors comprising the first double-stranded circular DNA vector and the second double-stranded circular DNA vector comprise the protelomerase gene sequence and the endonuclease gene sequence.

3. The transformed cell of claim 2, wherein the first double-stranded circular DNA vector comprises the protelomerase gene sequence in the other region between the pair of protelomerase recognition sequences, and the second double-stranded circular DNA vector comprises the endonuclease gene sequence.

4. The transformed cell of claim 2, wherein the first double-stranded circular DNA vector comprises the endonuclease gene sequence in the other region between the pair of protelomerase recognition sequences, and the second double-stranded circular DNA vector comprises the protelomerase gene sequence.

5. The transformed cell of claim 2, wherein the second double-stranded circular DNA vector comprises the protelomerase gene sequence and the endonuclease gene sequence.

6. The transformed cell of claim 2, wherein the transformed cell further comprises a third double-stranded circular DNA vector,
wherein the second double-stranded circular DNA vector comprises the protelomerase gene sequence, and
wherein the third double-stranded circular DNA vector comprises:
the endonuclease gene sequence; and
at least one endonuclease recognition sequence which is recognized and cleaved by the endonuclease or an active fragment thereof.

7. The transformed cell of claim 2, wherein
the second double-stranded circular DNA vector comprises the protelomerase gene sequence and the endonuclease gene sequence, and
the protelomerase gene sequence and the endonuclease gene sequence encode a fusion polypeptide comprising the protelomerase or an active fragment thereof and the endonuclease or an active fragment thereof.

8. The transformed cell of claim 1, wherein the first double-stranded circular DNA vector further comprises a spacer sequence placed between the endonuclease recognition sequence and the protelomerase recognition sequence.

9. The transformed cell of claim 1, wherein the protelomerase is selected from TelN protelomerase, TelA protelomerase, and TelK protelomerase.

10. The transformed cell of claim 1, wherein the endonuclease is a homing endonuclease.

11. The transformed cell of claim 10, wherein the homing endonuclease is selected from I-SceI homing endonuclease, I-CeuI homing endonuclease, and I-CreI homing endonuclease.

12. The transformed cell of claim 1, wherein the first double-stranded circular DNA vector and the second double-stranded circular DNA vector comprise antibiotic-resistance genes which provide resistance to different antibiotics.

13. The transformed cell of claim 1, wherein the first double-stranded circular DNA vector and the second double-stranded circular DNA vector are replicated by an identical replication mechanism.

14. The transformed cell of claim 13, wherein the first double-stranded circular DNA vector and the second double-stranded circular DNA vector comprise an identical replication initiation region.

15. The transformed cell of claim 1, wherein the transformed cell is selected from a bacterial cell and a eukaryotic cell.

16. A composition for transformation, comprising at least a first double-stranded circular DNA vector and a second double-stranded circular DNA vector,
wherein the first double-stranded circular DNA vector comprises:
a pair of protelomerase recognition sequences;
a nucleic acid sequence of interest which is placed in a region between the pair of protelomerase recognition sequences; and
at least one endonuclease recognition sequence which is placed in an other region between the pair of protelomerase recognition sequences, and
wherein the second double-stranded circular DNA vector comprises:
a protelomerase gene sequence which encodes a protelomerase or an active fragment thereof that recognizes and cleaves the pair of protelomerase recognition sequences and which is placed under the control of a promoter capable of controlling expression; and/or
an endonuclease gene sequence which encodes an endonuclease or an active fragment thereof that recognizes and cleaves the endonuclease recognition sequence and which is placed under the control of a promoter capable of controlling expression, and
wherein the second double-stranded circular DNA vector comprises at least one endonuclease recognition sequence which is recognized and cleaved by the endonuclease or an active fragment thereof.

17. A method for producing a linear covalently closed DNA, comprising:
culturing the transformed cell of claim 1;
inducing the expression of the protelomerase or an active fragment thereof and the endonuclease or an active fragment thereof in the transformed cell; and
extracting DNA from the transformed cell.

18. The method of claim 17, further comprising:
isolating the linear covalently closed DNA after extracting the DNA from the transformed cell.

19. The method of claim 17, further comprising:
introducing a composition for transformation to a host cell so as to produce a transformed cell comprising a first double-stranded circular DNA vector and a second double-stranded circular DNA vector, before culturing the transformed cell,
wherein the composition for transformation comprises at least the first double-stranded circular DNA vector and a second double-stranded circular DNA vector,
wherein the first double-stranded circular DNA vector comprises:
a pair of protelomerase recognition sequences;
a nucleic acid sequence of interest which is placed in a region between the pair of protelomerase recognition sequences; and
at least one endonuclease recognition sequence which is placed in an other region between the pair of protelomerase recognition sequences, and
wherein the second double-stranded circular DNA vector comprises:
a protelomerase gene sequence which encodes a protelomerase or an active fragment thereof that recognizes and cleaves the pair of protelomerase recognition sequences and which is placed under the control of a promoter capable of controlling expression; and/or
an endonuclease gene sequence which encodes an endonuclease or an active fragment thereof that recognizes and cleaves the endonuclease recognition sequence and which is placed under the control of a promoter capable of controlling expression, and
wherein the second double-stranded circular DNA vector comprises at least one endonuclease recognition sequence which is recognized and cleaved by the endonuclease or an active fragment thereof.

20. The method of claim 17, further comprising:
introducing the first double-stranded circular DNA vector to a host cell transformed with the second double-stranded circular DNA vector so as to produce the transformed cell comprising the first double-stranded circular DNA vector and the second double-stranded circular DNA vector, before culturing the transformed cell.
